(19) United States
(12) Patent Application Publication
(10) Pub. No.: US 2026/0294230 A1
(43) Pub. Date: Oct. 1, 2026

(54) METHODS, SYSTEMS, AND COMPUTER READABLE MEDIA FOR TESTING VISUAL FUNCTION USING VIRTUAL MOBILITY TESTS

(71) Applicant: The Trustees of the University of Pennsylvania, Philadelphia, PA (US)

(72) Inventors: Jean Bennett, Bryn Mawr, PA (US); Tomas Santiago Aleman, Croydon, PA (US); Manzar Ashtari, New Hyde Park, NY (US); Alexander Jacob Miller, Philadelphia, PA (US); Nancy Bennett, Los Angeles, CA (US)

(21) Appl. No.: 19/702,692

(22) Filed: Jun. 9, 2026

Related U.S. Application Data
(63) Continuation of application No. 17/079,119, filed on Oct. 23, 2020, now abandoned.
Application No. 17/079,119 is a continuation-in-part of application No. PCT/US19/29173, filed on Apr. 25, 2019.
(60) Provisional application No. 62/662,737, filed on Apr. 25, 2018.

(51) Int. Cl. A61B 3/00; A61B 3/02; G02B 27/01; G06F 3/01

(52) U.S. Cl. CPC A61B 3/0041; A61B 3/02; G02B 27/0172; G06F 3/013

(57) ABSTRACT

Methods, systems, and computer readable media for testing visual function using virtual mobility tests are disclosed. One system includes a processor and a memory. The system is configured for configuring a virtual mobility test for testing visual function of a user; generating the virtual mobility test; and analyzing a user’s performance during the virtual mobility test for determining the visual function of the user based on user interaction with objects in the virtual mobility test and using data from body movement detection sensors.

### PRIORITY CLAIM

This application is a continuation of U.S. Patent Application Serial No. 17/079,119, filed Oct. 23, 2020, which is a continuation-in-part of PCT International Patent Application Serial No. PCT/US19/29173, filed April 25, 2019, which claims the benefit of U.S. Provisional Patent Application Serial No. 62/662,737, filed April 25, 2018. The disclosures of these applications are incorporated herein by reference in their entireties.

### TECHNICAL FIELD

The subject matter described herein relates to virtual reality. More particularly, the subject matter described herein relates to methods, systems, and computer readable media for testing visual function using virtual mobility tests.

### BACKGROUND

One challenge with developing treatments for eye disorders involves developing test paradigms that can quickly, accurately, and reproducibly characterize the level of visual function and functional vision in real-life situations. Visual function can encompass many different aspects or parameters of vision, including visual acuity (resolution), visual field extent (peripheral vision), contrast sensitivity, motion detection, color vision, light sensitivity, and the pattern recovery or adaptation to different light exposures, to name a few. Functional vision, i.e., the ability to use vision to carry out different tasks, may therefore be considered a behavioral direct consequence of visual function. These attributes of vision are typically tested in isolation, e.g., in a scenario detached from the real-life use of vision. For example, a physical mobility test involving an obstacle course having various obstacles in a room may be used to evaluate one or more aspects of vision function. However, such a mobility test can involve a number of issues including time-consuming setup, limited configurability, risk of injury to users, and limited quantitation of results.

Accordingly, there exists a need for methods, systems, and computer readable media for testing visual function using virtual mobility tests.

### SUMMARY

Methods, systems, and computer readable media for testing visual function using virtual mobility tests are disclosed. One system includes a processor and a memory. The system is configured for receiving configuration information for setting up a virtual mobility test for testing visual function, generating the virtual mobility test; and analyzing performance of the user during the virtual mobility test for determining the visual function of the user based on user interaction with objects in the virtual mobility test using data from body movement detection sensors.

One method includes configuring a virtual mobility test for testing visual function of a user; generating the virtual mobility test; and analyzing performance of the user during the virtual mobility test for determining the visual function of the user based on user interaction with objects in the virtual mobility test using data obtained from body movement detection sensors.

The subject matter described herein may be implemented in hardware, software, firmware, or any combination thereof. As such, the terms “function” or “node” as used herein refer to hardware, which may also include software and/or firmware components, for implementing the feature(s) being described. In some exemplary implementations, the subject matter described herein may be implemented using a computer readable medium having stored thereon computer executable instructions that when executed by the processor of a computer, control the computer to perform steps. Exemplary computer readable media suitable for implementing the subject matter described herein include non-transitory computer readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms. In some exemplary implementations, the subject matter described herein may be implemented using hardware, software, firmware delivering augmented or virtual reality.

### BRIEF DESCRIPTION OF THE DRAWINGS

The subject matter described herein will now be explained with reference to the accompanying drawings of which:

FIG. 1 is a diagram illustrating an example virtual mobility test system (VMTS) for testing visual function;

FIG. 2 is a diagram illustrating an example template for a virtual mobility test;

FIG. 3 is a diagram illustrating a user performing a virtual mobility test;

FIG. 4 is a diagram illustrating example obstacles in a virtual mobility test;

FIG. 5 is a diagram illustrating various sized obstacles in a virtual mobility test;

FIG. 6 is a diagram illustrating virtual mobility tests with various lighting conditions;

FIG. 7 is a diagram illustrating example data captured during a virtual mobility test;

FIG. 8 is a diagram illustrating various aspects of an example virtual mobility test;

FIGS. 9A-9C depict graphs indicating various data gathered from subjects in a study using virtual mobility testing;

FIG. 10 is a flow chart illustrating an example process for testing visual function using a virtual mobility test;

FIG. 11 is a flow chart illustrating an example process for dissecting or analyzing two different parameters of visual function; and

FIG. 12 is a flow chart illustrating an example process for evaluating effectiveness of gene therapy on visual function of a user using virtual mobility tests.

### DETAILED DESCRIPTION

The subject matter described herein relates to methods, systems, and computer readable media for testing visual function using virtual mobility tests. A conventional mobility test for testing visual function of a user may involve one or more physical obstacle courses and/or other physical activities to perform. Such courses and/or physical activities may be based on real-life scenarios and/or activities, e.g., walking in a dim hallway or walking on a floor cluttered with obstacles. Existing mobility tests, however, have limited configurability and other issues. For example, conventional mobility tests are, by design, generally inflexible and difficult to implement and reproduce since these tests are usually designed using a particular implementation and equipment, e.g., a test designer’s specific hardware, obstacles, and physical space requirements.

One example of a ‘real-life’ or physical mobility test is a “RPE65” test for testing for retinal disease that affects ability to see in low luminance conditions, e.g., a retinal dystrophy due to retinal pigment epithelium 65 (RPE65) gene mutations. This physical test measures how a person functions in a vision-related activity of avoiding obstacles while following a pathway in different levels of illumination. While this physical test reflects the everyday life level of vision for RPE65-associated disease, the “RPE65” test suffers from a number of limitations. Example limitations for the “RPE65” test are discussed below.
1) The “RPE65” test is limited in usefulness for other populations of low vision patients. For example, the test cannot be used reliably to elicit visual limitations of individuals with fairly good visual acuity (e.g., 20/60 or better) but limited fields of vision.
2) The set-up of the “RPE65” test is challenging in that it requires a dedicated, large space. For example, the test area for the “RPE65” test must be capable of holding a 17 feet (ft) X 10 ft obstacle course, the test user (and companion) and the test operators, and cameras. Further, the room must be light-tight (e.g., not transmitting or reflecting light) and capable of presenting lighting conditions at a range of calibrated, accurate luminance levels (e.g., 1, 4, 10, 50, 125, 250, and 400 lux). Further, this illumination must be uniform in the test area.
3) Setting-up a physical obstacle course and randomizing assignment and positions of obstacles for the “RPE65” test (even for a limited number of layouts) is time-consuming.
4) Physical objects on a physical obstacle course are injury risk to patients (e.g., obstacles can cause a test user to fall or trip).
5) A “RPE65” test user can cheat during the test by using “echo-location” of objects instead of their vision to identify large objects.
6) A “RPE65” test user must be guided back to the course by the test operator if the user goes off course.
7) The “RPE65” test does not take into account that different individuals have different heights (and thus different visual angles).
8) The “RPE65” test captures video recordings of the subject’s performance which are then graded by outside consultants. This results in potential disclosure of personal identifiers.

The “RPE65” test has difficult and limited quantitation for evaluating a test user’s performance. For example, the scoring system for this test is challenging as it requires review of videos by masked graders and subjective grading of collisions and other aspects of the performance. Further, since the data is collected through videos showing the performance in two dimensions and focuses generally on the feet, there is no opportunity to collect additional relevant data, such as direction of gaze, likelihood of collision with objects beyond the view of the camera lens, velocity in different directions, acceleration, etc.

In accordance with some aspects of the subject matter described herein, techniques, methods, systems, or mechanisms are disclosed for using a virtual (e.g., virtual reality (VR) based) mobility test. For example, a virtual mobility test system (e.g., a computer, a VR headset, and body movement detection sensors) may configure, generate, and analyze a virtual mobility test for testing visual function of a user. In this example, the test operator or the virtual mobility test system may change virtually any aspect of the virtual mobility test, including, for example, size, shape, and placement of obstacles, lighting conditions, and may provide haptic and audio user feedback, and may use these capabilities to test various different diseases and/or eye or vision conditions. Moreover, since a virtual mobility test does not involve real or physical obstacles, cost and time associated with setting up and administering the virtual mobility test may be significantly reduced compared to a physical mobility test. Further, a virtual mobility test may be configured to efficiently capture and store relevant data not obtained in conventional physical tests (e.g., eye or head movements) and/or may capture data with more precision (e.g., via body movement detection sensors) than in conventional physical tests. With the VR system, the scene can be displayed to one eye or the other or to both eyes simultaneously. Furthermore, with additional and more precise data, a virtual mobility test system or a related entity may produce more objective and/or accurate test results (e.g., user performance scores).

In accordance with some aspects of the subject matter described herein, techniques, methods, systems, or mechanisms are disclosed for evaluating (e.g., detecting and/or quantifying) the effectiveness of gene therapy on visual function of a user using a virtual mobility test or a related test system.

Reference will now be made in detail to exemplary embodiments of the subject matter described herein, examples of which are illustrated in the accompanying drawings. Wherever possible, the same reference numbers may be used throughout the drawings to refer to the same or like parts.

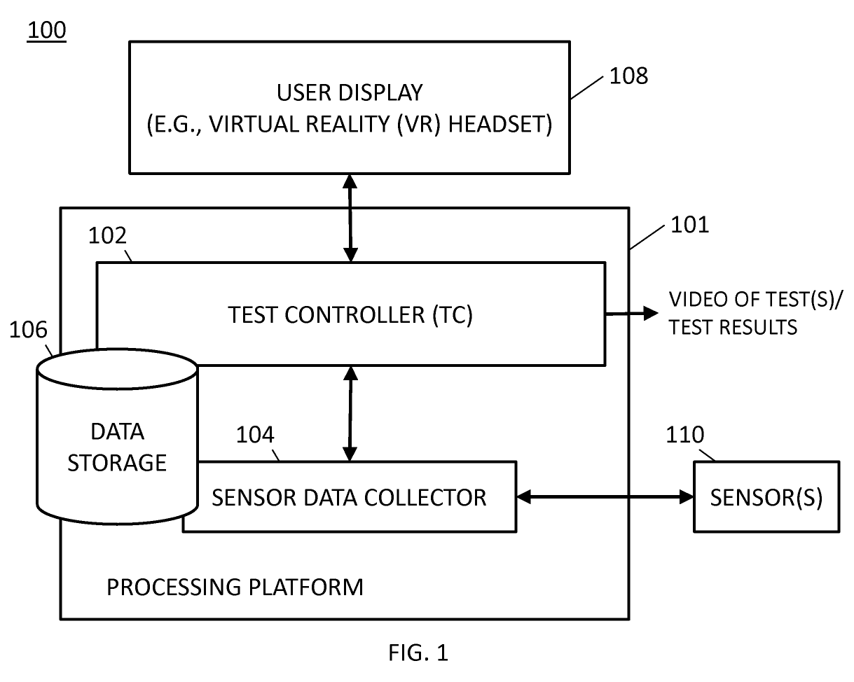

FIG. 1 is a diagram illustrating an example virtual mobility test system (VMTS) 100 for testing visual function. In FIG. 1, virtual mobility test system (VMTS) 100 may include a processing platform 101, a user display 108, and one or more sensors 110 are depicted. VMTS 100 may represent any suitable entity or entities (e.g., a VIVE virtual reality (VR) system, one or more servers, a desktop computer, a phone, a tablet computer, or a laptop) for testing visual function in a virtual (e.g., VR) environment. For example, VMTS 100 may be a laptop or a desktop computer executing one or more applications and may interact with various modules or components therein. In some embodiments, VMTS 100 may include a communications interface for receiving configuration information associated with generating or setting up a virtual environment for testing various aspects of visual function and/or detecting whether a user (e.g., a test participant or subject) may be displaying symptoms or characteristics of one or more eye issues or related conditions. In some embodiments, VMTS 100 may include one or more communications interfaces for receiving sensor data or feedback from one or more physical sensors 110 associated with a test user. For example, sensors 110 may detect body movement (e.g., of feet, arms, and head), along with related characteristics (e.g., speed and/or direction of movement). In some embodiments, VMTS 100 may include one or more processors, memories, and/or other hardware for generating a virtual environment, displaying a virtual environment (e.g., including various user interactions with the environment and related effects caused by the interactions, such as collisions with virtual obstacles, movements that the user makes to avoid collision (such as lifting up a leg to step over an object or ducking under a sign) or purposeful touch such as stepping on a “step” or touching a finish line), and recording or storing test output and related test results (e.g., including text based logs indicating sensor data and/or a video recreation of the test involving a user’s progress during the test).

In some embodiments, VMTS 100 may utilize processing platform 101 for providing various functionality. Processing platform 101 may represent any suitable entity or entities (e.g., one or more processors, computers, nodes, or computing platforms) for implementing various modules or system components. For example, processing platform 101 may include a server or computing device containing one or more processors and memory (e.g., flash, random-access memory, or data storage). In this example, various software and/or firmware modules may be implemented using the hardware at processing platform 101. In some embodiments, processing platform 101 may be communicatively connected to user display 108 and/or sensors 110.

In some embodiments, VMTS 100 or processing platform 101 may include a test controller (TC) 102, a sensor data collector 104, and a data storage 106. TC 102 may represent any suitable entity or entities (e.g., software executing on one or more processors) for performing one or more aspects associated with visual function testing in a virtual environment. For example, TC 102 may include functionality for configuring and generating a virtual environment for testing visual function of a user. In this example, TC 102 may also be configured for executing a related mobility test, providing output to user display 108 (e.g., a virtual reality (VR) display) or other device, receiving input from one or more sensors 110 (e.g., accelerometers, gyroscopes, eye trackers, or other body movement sensing devices) or other devices (e.g., video cameras). Continuing with this example, TC 102 may be configured to analyze various input associated with a virtual mobility test and provide various metrics and test results, e.g., a virtual recreation or replay of a user performing the virtual mobility test.

In some embodiments, TC 102 may communicate or interact with user display 108. User display 108 may represent any suitable entity or entities for receiving and providing information (e.g., audio, video, and/or haptic feedback) to a user. For example, user display 108 may include a VR headset (e.g., a VIVE VR headset), glasses, a mobile device, and/or another device that includes software executing on one or more processors. In this example, user display 108 may include various communications interfaces capable of communicating with TC 102, VMTS 100, sensors 110, and/or other entities. In some embodiments, TC 102 or VMTS 100 may stream data for displaying a virtual environment to user display 108. For example, TC 102 or VMTS 100 may receive input during testing from various sensors 110 related to a user’s progress through a mobility test (e.g., obstacle course) in the virtual environment and may send data (e.g., in real-time or near real-time) to reflect or depict a user’s progress along the course based on a variety of factors, e.g., a preconfigured obstacle course map and user interactions or received feedback from sensors 110.

In some embodiments, TC 102 may communicate or interact with sensor data collector 104. Sensor data collector 104 may represent any suitable entity or entities (e.g., software executing on one or more processors and/or one or more communications interfaces) for receiving or obtaining sensor data and/or other information from sensors 110 (e.g., body movement detection sensors). For example, sensor data collector 104 may include an antenna or other hardware for receiving input via wireless technologies, e.g., Wi-Fi, Bluetooth, etc. In this example, sensor data collector 104 may be capable of identifying, collating, and/or analyzing input from various sensors 110. In some embodiments, sensors 110 may include accelerometers and/or gyroscopes to detect various aspects of body movements. In some embodiments, sensors 110 may include one or more surface electrodes attached to the skin of a user and sensor data collector 104 (or TC 102) may analyze and interpret EMG data into body movement.

In some embodiments, sensors 110 or related components may be part of an integrated and/or wearable device, such as a VR display, a wristband, armband, glove, leg band, sock, headband, mask, sleeve, shirt, pants, or other device. For example, sensors 110 may be located at or near user display 108. In this example, such sensors 110 may be configured to identify or track eye movement, squinting, pupil changes, and/or other aspects related to eyesight.

In some embodiments, VMTS 100 or one or more modules therein (e.g., TC 102 and/or sensor data collector 104) may provide functionality for tailoring a virtual mobility test (e.g., a mobility test in a virtual environment using a VR system) to an individual and/or an eye or vision condition or disease. For example, a virtual mobility test as described herein may be administered such that each eye is used separately or both eyes are used together. In this example, by using this ability, the progression of an eye disease or the impact of an intervention can be measured according to the effects on each individual eye. In many eye diseases, there is symmetry between the eyes. Thus, if an intervention is tested in one eye, the other eye can serve as an untreated control and the difference in the performance between the two eyes can be used to evaluate safety and efficacy of the intervention. For example, data can be gathered from monocular (e.g., single eye) tests on the user’s perspective (e.g., is one object in front of another) and from binocular (e.g., two eyes) tests on the user’s depth perception and stereopsis.

In some embodiments, VMTS 100 or one or more modules therein may configure a virtual mobility test for monitoring eye health and/or related vision over a period of time. For example, changes from a baseline and/or changes in one eye compared to the other eye may measure the clinical utility of a treatment in that an increase in visually based orientation and mobility skills increases an individual’s safety and independence. Further, gaining the ability to orient and navigate under different conditions (e.g., using lower light levels than previously possible) may reflect an improvement of those activities of daily living that depend on vision.

In some embodiments, VMTS 100 or one or more modules therein may perform virtual mobility tests for a variety of purposes. For example, a virtual mobility test may be used for rehabilitation purposes (e.g., as part of exercises that can potentially improve the use of vision function or maintain existing vision function). In another example, a virtual mobility test may also be used for machine learning and artificial intelligence purposes.

In some embodiments, a virtual mobility test may be configured (e.g., using operator preferences or settings) to include content tailored to a particular vision condition or disease. In some embodiments, the configured content may be usable to facilitate or rule out a diagnosis and may at least in part be based on known symptoms associated with a particular vision condition. For example, there are different deficits in different ophthalmic diseases ranging from light sensitivity, color detection, contrast perception, depth perception, focus, movement perception, etc. In this example, a virtual mobility test may be configured such that each of these features can be tested, e.g., by controlling these variables (e.g., by adjusting lighting conditions and/or other conditions in the virtual environment where the virtual mobility test is to occur).

In some embodiments, a virtual mobility test may be configured to measure and/or evaluate symptoms of one or more retinal diseases, vision conditions, or related issues. For example, VMTS 100 or one or more modules therein may use predefined knowledge of symptoms regarding a vision condition to generate or configure a virtual mobility test for measuring and/or evaluating aspects of those symptoms. In this example, when generating or configuring a virtual mobility test for measuring and/or evaluating certain symptoms, VMTS 100 or one or more modules may query a data store using the predefined symptoms to identify predefined tasks and/or course portions usable for measuring and/or evaluating those symptoms. Example retinal different retinal diseases, vision conditions, or related issues that a virtual mobility test may be configured for include macular degeneration, optic nerve disease, retinitis pigmentosa (RP), choroideremia (CHM), one or more forms of color blindness, blue cone monochromacy, achromatopsia, diabetic retinopathy, retinal ischemia, or various central nervous system (CNS) disorders that affect vision.

In some embodiments, a virtual mobility test for measuring symptoms of macular degeneration (e.g., age-related macular degeneration, Stargardt disease, cone-rod dystrophy, etc.) may be configured and administered using VMTS 100 or one or more modules therein. A macular degeneration related virtual mobility test may be configured for measuring one or more aspects of macula function, e.g., visual acuity, color discrimination, and/or contrast sensitivity. In some examples, a macular degeneration related virtual mobility test may use arrows (e.g., directional course arrows) to measure visual acuity of the user, e.g., arrows may initially be designated to be visible with 20/200 Snellen visual acuity (e.g., low vision) but the arrows can be made smaller or larger to reflect the sizes normally measured on the visual acuity early treatment diabetic retinopathy study (ETDRS) chart (e.g., 20/15, 20/20, 20/25, 20/40, 20/50, 20/63, 20/80, 20/100, 20/200). In some examples, a macular degeneration related virtual mobility test may involve modifying colors of arrows, background, and/or objects to measure color discrimination because with macular disease often detection of lower wavelengths of light (e.g., shades of yellow, purple, and pastels) is the first to be impaired in the disease process. As the disease progresses, all color perception may be lost leaving the person able to discriminate only signals mediated by rod photoreceptors (e.g., shades of grey). In order to elicit color perception abilities, arrows of one color can be placed over a background of another color similar to how numbers are presented in an Ishihara color vision test, e.g., using pseudoisochromatic plates. In some examples, a macular degeneration related virtual mobility test may present objects in colors that contrast with the color of the background. The contrast of the arrows and objects and shading of their edges in a virtual mobility test may also be modified to measure contrast discrimination because individuals with macular disease often have trouble identifying faces, objects, and obstacles due to their impaired ability to detect contrast.

In some embodiments, a virtual mobility test for measuring symptoms of optic nerve disease (e.g., glaucoma, optic neuritis, mitochondrial disorders such as Leber’s hereditary optic neuropathy) may be configured and administered using VMTS 100 or one or more modules therein. Because of the known symptoms of optic nerve disease, an optic nerve disease related virtual mobility test may be configured to measure and/or evaluate visual fields and light sensitivity of a user. In some examples, an optic nerve disease related virtual mobility test may present swinging objects from different directions and the test may measure or evaluate a user’s ability to detect these objects (e.g., by avoidance of collisions) while navigating the mobility course. The swinging objects in the test may be shown at different sizes in order to further elicit and evaluate the user’s ability to use peripheral vision. In some examples, an optic nerve disease related virtual mobility test may present swinging objects with different luminances in order to measure changes in light sensitivity associated with disease progression. The brighter lights may be perceived by users with optic never disease as having halos, which may have an impact on the user’s avoidance of the swinging objects. The user can be asked beforehand to report any perception of halos around lights and can be documented and used for review or test analysis. In some examples, an optic nerve disease related virtual mobility test may present swinging objects with different levels of contrast, in order to measure changes in contrast sensitivity associated with disease progression. In some examples, an optic nerve disease related virtual mobility test may involve using brightness and/or luminance of arrows and objects in a related mobility course to measure brightness discrimination.

In some embodiments, a virtual mobility test for measuring symptoms of RP in any of its various forms (e.g., RP found in a syndromic disease such as Usher Syndrome, Bardet-Biedl, Joubert, etc.) may be configured and administered using VMTS 100 or one or more modules therein. Symptoms of RP can include loss of peripheral vision followed eventually by loss of central vision and night blindness. Depending on the stage of disease, an RP related virtual mobility test may include a similar protocol established for the RPE65 form of Leber’s congenital amaurosis. In some embodiments, e.g., in order to further evaluate peripheral vision, an RP related virtual mobility test may present swinging objects from different directions, with different sizes, and different luminance (as described above for optic nerve disease testing). For further analyses of light sensitivity, an RP related virtual mobility test may involve a user using hand tracking and eye-tracking to control dimmer switches for virtual lights, where the user may set the brightness of the lights to where they think that they see best. The user’s perceptions of the conditions in which they see best can be compared with results measured using a standardized test.

In some embodiments, a virtual mobility test for measuring symptoms of CHM may be configured and administered using VMTS 100 or one or more modules therein. A CHM related virtual mobility test may be configured differently depending on the stage of the disease and/or age of the user (e.g., the person performing the test). For example, when testing juveniles with CHM, a CHM related virtual mobility test may be configured to focus on evaluating a user’s light sensitivity, e.g., similar to the test described in Appendix A. However, since individuals with CHM usually have good visual acuity, arrows in a CHM related virtual mobility test may be small in size (on the order of 20/15 Snellen visual acuity). As the disease progresses, individuals with CHM may lose their visual fields and may also suffer from glare and difficulties in equilibrating if light levels are rapidly changed. Therefore, a CHM related virtual mobility test may present a number of swinging objects (e.g., to probe visual field loss), and lights that flash at designated intervals (e.g., to mimic glare and changes in light levels). In some embodiments, in lieu of swinging objects, a CHM related virtual mobility test may include objects and/or situations that might take place in daily life, e.g., birds flying overhead or a tree branch swaying in the breeze. Such situations may allow the user to position themselves such that glare is blocked by the flying object. In some embodiments, user interactions with daily life situations may act as a game or provide a sense of play. In such embodiments, VMTS 100 or one or more modules therein may use eye tracking to measure user response objectively with regard to interactive situations. In some examples, a CHM related virtual mobility test may involve dimming lighting and/or altering contrast levels at prescribed intervals or aperiodically (e.g., randomly).

In some embodiments, a virtual mobility test for measuring symptoms of red-green color blindness may be configured and administered using VMTS 100 or one or more modules therein. A red-green color blindness related virtual mobility test may focus on measuring or evaluating a user’s ability to discern or detect different colors, e.g., red and green. In some examples, a red-green color blindness related virtual mobility test may present arrows in green on a red background (or vice versa; red arrows on a green background). In addition to or alternatively, in some examples, a red-green color blindness related virtual mobility test may present obstacles as red objects on a green background.

In some embodiments, a virtual mobility test for measuring symptoms of blue-yellow color blindness may be configured and administered using VMTS 100 or one or more modules therein. A blue-yellow color blindness related virtual mobility test may focus on measuring or evaluating a user’s ability to discern or detect different colors, e.g., blue and yellow. In some examples, a blue-yellow color blindness related virtual mobility test may present arrows in blue on a yellow background (or vice versa; yellow arrows on a blue background). In addition to or alternatively, in some examples, a blue-yellow color blindness related virtual mobility test may present obstacles as yellow objects on a blue background.

In some embodiments, a virtual mobility test for measuring symptoms of blue cone monochromacy may be configured and administered using VMTS 100 or one or more modules therein. A blue cone monochromacy related virtual mobility test may focus on measuring or evaluating a user’s ability to discern or detect different colors. In some examples, a blue cone monochromacy related virtual mobility test may involve a virtual course or portion thereof being presented in greyscale for testing more detail of what the user sees. In addition to or alternatively, in some examples, a blue cone monochromacy related virtual mobility test may involve a virtual course or portion thereof being presented one color or two different colors. In some example where two colors are used, a blue cone monochromacy related virtual mobility test may present arrows in blue on a yellow background (or vice versa; yellow arrows on a blue background) or arrows in blue on a yellow background (or vice versa; yellow arrows on a blue background). In addition to or alternatively, in some examples, a blue cone monochromacy related virtual mobility test may present obstacles as yellow objects on a blue background or obstacles as red objects on a green background. In some embodiments, VMTS 100 or one or more modules therein may compare the user’s performances between the differently colored courses, e.g., change in performance from the greyscale course to the blue-yellow course.

In some embodiments, a virtual mobility test for measuring symptoms of achromatopsia may be configured and administered using VMTS 100 or one or more modules therein. Individuals with achromatopsia may suffer from sensitivity to lights and glare, have poor visual acuity, and impaired color vision, e.g., they may only see objects in shades of grey and black and white. In some examples, instead of starting to present a mobility course with dim light (e.g., as with a RPE65-LCA related test), an achromatopsia related virtual mobility test may initially present a mobility course with bright light and then subsequent testing may test whether the user can perform more accurately at dimmer light, e.g., by decreasing brightness in subsequent runs. In some examples, an achromatopsia related virtual mobility test may determine a the threshold lighting value at which a user is able to perform the test accurately, e.g., complete a related mobility course with an acceptable number of collisions, such less than two collisions. In some examples, an achromatopsia related virtual mobility test may involve a user using hand tracking and eye-tracking to control dimmer switches for virtual lights, where the user may set the brightness of the lights to where they think that they see best. The user’s perceptions of the conditions in which they see best can be compared with results measured using a standardized test. In some examples, an achromatopsia related virtual mobility test may present arrows and/or obstacles in selected color combinations similar to that described for red-green color blindness or in blue cone monochromacy.

In some embodiments, a virtual mobility test for measuring symptoms of diabetic retinopathy may be configured and administered using VMTS 100 or one or more modules therein. Symptoms of diabetic retinopathy can include blurred vision, impaired field of view, difficulty with color discrimination. In some examples, a diabetic retinopathy related virtual mobility test may use arrows (e.g., directional course arrows) to measure visual acuity of the user, e.g., arrows may initially be designated to be visible with 20/200 Snellen visual acuity (e.g., low vision) but the arrows can be made smaller or larger to reflect the sizes normally measured on the visual acuity ETDRS chart. In some examples, e.g., in one or more iterations of a diabetic retinopathy related virtual mobility test, a user could be provided a virtual dial that they can spin to optimize focus. Their perceived optimal focus could be compared with what is measured using a standardized test or other testing. In some examples, a diabetic retinopathy related virtual mobility test may involve modifying colors of arrows, background, and/or objects to measure color discrimination. In order to elicit color perception abilities, arrows in a diabetic retinopathy related virtual mobility test of one color can be placed over a background of another color similar to how numbers are presented in an Ishihara color vision test, e.g., using pseudoisochromatic plates. In some examples, a diabetic retinopathy related virtual mobility test may present objects in colors that contrast with the color of the background. In some examples, VMTS 100 or one or more modules therein may utilize haptic feedback with a diabetic retinopathy related virtual mobility test, e.g., by providing vibrations when a user approaches objects or obstacles. In such examples, haptic feedback or other audio components can be utilized with a diabetic retinopathy related virtual mobility test for testing whether a user utilizes echo-location (spatial audio) in their daily life.

In some embodiments, a virtual mobility test for measuring symptoms of retinal ischemia may be configured and administered using VMTS 100 or one or more modules therein. Symptoms of retinal ischemia can include blurred vision, graying or dimming of vision and/or loss of visual field. In some examples, a retinal ischemia related virtual mobility test may use arrows (e.g., directional course arrows) to measure visual acuity of the user, e.g., arrows may initially be designated to be visible with 20/200 Snellen visual acuity (e.g., low vision) but the arrows can be made smaller or larger to reflect the sizes normally measured on the visual acuity ETDRS chart. In some examples, a retinal ischemia related virtual mobility test may involve modifying colors of arrows, background, and/or objects to measure color discrimination. In order to elicit color perception abilities, arrows in a retinal ischemia related virtual mobility test of one color can be placed over a background of another color similar to how numbers are presented in an Ishihara color vision test, e.g., using pseudoisochromatic plates. In some examples, a retinal ischemia related virtual mobility test may present objects in colors that contrast with the color of the background. In some examples, a retinal ischemia related virtual mobility test may present a number of swinging objects (e.g., to probe visual field loss).

In some embodiments, a virtual mobility test for measuring symptoms of vision-affecting CNS disorders (e.g., a stroke or a brain tumor) may be configured and administered using VMTS 100 or one or more modules therein. Vision-affecting CNS disorders can result in vision observed only on one side for each eye or for both eyes together. As such, in some examples, a vision-affecting CNS disorder related virtual mobility test may involve testing various aspects associated with vision fields of a user. In some examples, a vision-affecting CNS disorder related virtual mobility test may present swinging objects from different directions and the test may measure or evaluate a user’s ability to detect these objects (e.g., by avoidance of collisions) while navigating the mobility course. The swinging objects in the test may be shown at different sizes in order to further elicit and evaluate the user’s ability to use peripheral vision.

In some embodiments, a virtual mobility test may be configured to include obstacles that represent challenges an individual can face in daily life, such as doorsteps, holes in the ground, objects that jut in a user’s path, objects at various heights (e.g., waist high, head high, etc.), and objects which can swing into the user’s path. In such embodiments, risk of injury may be significantly reduced relative to a conventional mobility test since the obstacles in the virtual mobility test are virtual and not real.

In some embodiments, virtual obstacles (e.g., obstacles in a virtual mobility test or a related virtual environment) can be adjusted or resized dynamically or prior to testing. For example, virtual obstacles, as a group or individually, may be enlarged or reduced by a certain factor (50%) via a test operator and/or VMTS 100. In this example, a virtual mobility test may be configured to include dynamic obstacles that increase or decrease in size, e.g., if a user repeatedly hits the obstacle or cannot move past the obstacle.

In some embodiments, a virtual mobility test or a related obstacle course therein may be adjustable based on a user’s profile or related characteristics, e.g., height, weight, fitness level, age, or known deficiencies. For example, scalable obstacle courses may be useful for comparisons of performance of individuals who differ in height as user’s height (e.g., distance of the eyes to the objects on the ground) affects visual resolution (e.g., visual acuity). In another example, scalable obstacle courses may be useful for following the visual performance of a child over time, e.g., as the child will grow and become an adult. In some embodiments, scaling an obstacle course may also be useful to ensure that obstacles or elements in the virtual environment (e.g., tiles that make of a course segments) are sized appropriately (e.g., so that a user’s foot can fit along an approved path through the virtual obstacle course).

In some embodiments, a virtual mobility test or a related obstacle course therein may be adjustable so as to avoid or mitigate learning bias. In such embodiments, adjustment or modification may be performed such that a particular skill level or complexity for the test or course is maintained or represented. For example, VMTS 100 may adjust a path and/or various locations of obstacles presented in a virtual mobility test so as to prevent or mitigate learning bias by a user. In this example, VMTS 100 may utilize an algorithm so that the modified virtual mobility test is substantially equivalent to an original virtual mobility test. In some embodiments, to achieve equivalence, VMTS 100 may utilize a ‘course shuffling’ algorithm that ensures the modified virtual mobility test includes similar number and types of obstacles, number and types of tasks, path complexity, and luminance levels as an initial virtual mobility test.

In some embodiments, a virtual mobility test or a related obstacle course therein may be configured, generated, or displayed based on various configurable settings. For example, a test operator may input or modify a configuration files with various settings. In this example, VMTS 100 or one or more modules therein may use the settings to configure, generate, and/or display the virtual mobility test or a related obstacle course therein.

In some embodiments, VMTS 100 or one or more modules therein may configure a virtual mobility test for testing a user’s vision function in a variety of lighting conditions. For example, light levels utilized for a virtual mobility test may be routinely encountered in day-to-day situations, such as walking through an office building, crossing a street at dusk, or locating objects in a dimly-lit restaurant.

In some embodiments, VMTS 100 or one or more modules therein may adjust lighting conditions for a virtual environment or related obstacle course. In this example, VMTS 100 or one or more modules therein may adjust luminance of obstacles, path arrows, hands and feet, finish line, and/or floor tiles associated with the virtual environment or related obstacle course. In another example, VMTS 100 or one or more modules therein may design aspects (e.g., objects, obstacles, terrain, etc.) of the virtual environment to minimize light bleeding and/or other issues that can affect test results (e.g., by using Gaussian textures on various virtual obstacles or other virtual objects).

In some embodiments, a virtual mobility test may be configured such that various types of user feedback are provided to a user. For example, three-dimensional (3-D) spatial auditory feedback may be provided to a user (e.g., via speakers associated with user display 108 or VMTS 100) when the user collides with an obstacle during a virtual mobility test. In this example, the auditory feedback may emulate a real-life sound or response (e.g., a ‘clanging’ sound or a ‘scraping’ sound depending on the obstacle, or a click when the user climbs up a “step”) and may be usable by the user to correct their direction or movements. In another example, haptic feedback may be provided to a user (e.g., via speakers associated with user display 108 or VMTS 100) when the user goes off-course (e.g., away from a designated path) in the virtual environment. In this example, by using haptic feedback, the user can be made aware of this occurrence without requiring a test operator to physically guide them back on-course and can also test whether the user can self-redirect appropriately without assistance.

In some embodiments, VMTS 100 or one or more modules therein (e.g., TC 102 and/or sensor data collector 104) may analyze various data associated with a virtual mobility test. For example, VMTS 100 or modules therein may record a virtual mobility user’s performance using sensors 110 and/or one or more video cameras. In this example, the data captured may be measured and analyzed using quantitative analysis (e.g., based on objective criteria). In some embodiments, in contrast to conventional mobility test, there may be little to no subjective interpretation of the performance. For example, from the start to the finish of a virtual mobility test (e.g., timed from when the virtual environment or scene is displayed until the user touches a finish flag at the finish line), details of each collision, details of movement of the user’s head, hands, and feet may be recorded and analyzed. In some embodiments, additional sensors (e.g., eyesight trackers and/or other devices) may be used to detect and record movements of other parts of the body.

In some embodiments, an obstacle in a virtual mobility test may include an object adjacent to a user’s path (e.g., a rectangular object, a hanging sign, a floating object), a black tile or an off-course (e.g., off-path) area, a “push-down” or “step-down” object that a user must press or depress, (otherwise there is a penalty for collision or avoidance of this object), or an object on the user’s path that needs to be stepped over.

In some embodiments, data captured digitally during testing may be analyzed for performance of the user. For example, the time before taking the first step, or the time necessary to complete a virtual mobility test and the number of errors (e.g., bumping into obstacles, using feet to ‘feel’ one’s way, and/or going off course and then correcting themselves after receiving auditory feedback) or the attempt of the user to correct themselves after they have collided with an obstacle may all be assessed to develop a composite analysis metric or score. In some embodiments, an audiotape and/or videotape may be generated during a virtual mobility test. In such example, digital records (e.g., sensor data or related information) and the audiotape and/or videotape may comprise source data for analyzing a user’s performance.

In some embodiments, VMTS 100 or related entities may score or measure a user’s performance during a virtual mobility test by using one or more scoring parameters. Some example scoring parameters may include a collision penalty may be assigned each time a particular obstacle is bumped or a score penalty for each obstacle bumped (even if an obstacle is bumped multiple times); an off-course penalty may be assigned if both feet are on tile(s) that do not have arrows or if the user bypasses tiles with arrows on the course (if one foot straddles the border of an adjacent tile or if the user steps backward on the course to take a second look, this may not considered off-course); a guidance penalty may be assigned if a user needs to be directed back on course by the test giver (or the virtual environment).

In some embodiments, VMTS 100 or related entities (e.g., a data storage 106, sensor data collector 104, or external device) may store test data and/or record a user’s performance in a virtual mobility test. For example, VMTS 100 or another element may record a user’s progress by recording frame by frame movement of head, hands, and feet using data from one or more sensors 110. In some embodiments, data associated with each collision between a user and an obstacle may be recorded and/or captured. In such embodiments, a captured collision may include data related to bodies or items involved, velocity of the body part(s) (e.g., head, foot, arm, etc.) involved in the collision, acceleration of the body part(s) (e.g., head, foot, arm, etc.) involved in the collision, the point of impact, the time and/or duration of impact, and scene or occurrence playback (e.g., the playback may include a replay (e.g., a video) of an avatar (e.g., graphics representing the user or body parts thereof) performing the body part movements that cause the collision).

In some embodiments, administering a virtual mobility test may include a user (e.g., a test participant) and one or more test operators, observers, or assistants. For example, a virtual mobility test may be conducted by a study team member and a technical assistant. The study team member may alternatively both administer the test and may monitor equipment used in the testing. The study team member may be present to help the user with course redirects or physical guidance, if necessary. The test operators, observers, and/or assistants may not give instructions or advise during the virtual mobility test. In some embodiments, a virtual mobility test may be conducted on a level floor in a space appropriate for the test, e.g., in a room with clearance of a 12 feet (ft) x 7 ft rectangular space, since the test may include one or more courses that require a user to turn in different directions and avoid obstacles of various sizes and heights along the way.

In some embodiments, before administering a virtual mobility test the virtual mobility test may be described to the user and the goals of the test may be explained (e.g., complete the course(s) as accurately and as quickly as possible). The user may be instructed to do their best to avoid all of the obstacles except for the steps, and to stay on the path. The user may be encouraged to take their time and focus on accuracy. The user may be reminded not only to look down for guidance arrows showing the direction to walk, but also to scan back and forth with their eyes so as to avoid obstacles that may be on the ground or at any height up to their head.

In some embodiments, a user may be given a practice session so that they understand how to use equipment, recognize guidance arrows that must be followed, are familiar with the obstacles and how to avoid or overcome them (e.g., how to step on the “push down” obstacles), and also how to complete the virtual mobility test (e.g., by touching a finish flag to mark completion of the test). The user may be reminded that during the official or scored test, that the course may be displayed to one eye or the other or to both eyes. The user may be told that they will not receive directions while the test is in progress. However, under certain circumstances (e.g., if the user does not know which way to go and pauses for more than 15 seconds, the tester or an element of the virtual mobility test (e.g., flashing arrows, words, sounds, etc.) may recommend that the user chooses a direction. The tester may also assist and/or assure the user regarding safety issues, e.g., the tester may stop the user if a particular direction puts the user at risk of injury.

In some embodiments, a user may be given one or more different practice tests (e.g., three tests or as many as are necessary to ensure that the user understands how to take the test). A practice test may use one or two courses that are different from courses used in the non-practice tests (e.g., tests that are scored) to be given. The same practice courses may be used for each user. The practice runs of a user may be recorded; however, the practice runs may not be scored.

In some embodiments, when a user is ready for an official (e.g., scored) virtual mobility test, the user may be fitted with user display 108 (e.g., a VR headset) and sensors 110 (e.g., body movement detection sensors). The user may also be dark adapted prior to the virtual mobility test. The user may be led to the virtual mobility test origin area and instructed to begin the test once the VR scene (e.g., the virtual environment) is displayed in user display 108. Alternatively, the user may be asked to move to a location containing a virtual illuminated circle on the floor which, when the test is illuminated, will become the starting point of the test. The onset of VR scene in user display 108 may mark the start of the test. During the test, an obstacle course may be traversed first with one eye “patched” or unable to see the VR scene (e.g., user display 108 may not show visuals on the left (eye) display, but show visuals on the right (eye) display), then the other eye “patched”, then both eyes “un-patched” or able to see the VR scene (e.g., user display 108 may show visuals on both the left and the right (eye) displays). The mobility test may involve various iterations of an obstacle course at different light intensities (e.g., incrementally dimmer or brighter), and at different layouts or configurations of elements therein (e.g., the path taken and the obstacles along the path may be changed after each iteration. For example, each obstacle course attempted by a user may have the same number of guidance arrows, turns, and obstacles, but to preclude a learning effect or bias, each attempt by the user may be performed using a different iteration of the obstacle course.

In some embodiments, a virtual mobility test or a related test presentation (e.g., administration) may be generated or modified for various purposes, e.g., data capture, data analysis, and/or educational purposes. For example, VMTS 100 or one or more modules therein may generate and administer a virtual mobility test that mimics a given vision condition. In this example, mimicking a vision condition may involve affecting a presentation of a virtual mobility course, e.g., blurring and/or dimming a virtual scene shown in a VR headset so that a user with normal sight (e.g., 20/20 vision and no known vision conditions) experiences symptoms of the vision condition. In this example, VMTS 100 or one or more modules therein may use results from the affected tests to generate ‘condition-affected’ baseline results obtained using normally-sighted users and/or for educating people about certain vision conditions.

In some embodiments, a virtual mobility test or a related test presentation may be generated or modified for diagnosing specific vision disorders. In some examples, a virtual mobility test or a related test presentation (e.g., administration) may diagnose forms of RP or Leber’s congenital amaurosis by testing a user’s performance using a virtual mobility course under different levels of luminance. In some examples, a virtual mobility test or a related test presentation (e.g., administration) may precisely measure how much red, green, and/or blue needs to be present (e.g., in a virtual object or background) to be detectable by a user and may use this measurement for diagnosing color blindness, achromatopsia or other disorders of central vision. In such examples, the test or presentation may involve adjusting levels of red, green, and blue light and/or altering colors of obstacles or backgrounds during testing.

In some embodiments, a virtual mobility test or a related test presentation may be generated or modified for characterizing loss of peripheral vision (e.g., in individuals with glaucoma or RP) by testing different degrees peripheral vision only in the goggles (e.g., with no central vision). By incorporating eye-tracking and adding progressively more areas to view, a virtual mobility test or a related test presentation may determine the exact extent of peripheral field loss. In some examples,. a peripheral vision related test or presentation may include an option for making virtual walls encroach upon or expand about a user depending on their performance on a given test. This may be analogous to a “staircase testing fashion” usable for measuring light sensitivity in the RPE65 form of Leber’s congenital amaurosis (except that it is applied to visual fields).

In some embodiments, a virtual mobility test or a related test presentation may be generated or modified for characterizing nystagmus (e.g., abnormal rotatory eye movements found in a number of vision disorders including Leber’s congenital amaurosis, ocular albinism, use of certain drugs, neurologic conditions) by using pupil-tracking to measure the amplitude of nystagmus and changes in amplitude associated with gaze or field of view. Nystagmus is associated with loss of visual acuity and so characterization and identification of nystagmus may lead to treatments which dampen nystagmus and thus improve vision.

In some embodiments, a virtual mobility test or a related test presentation may be generated or modified for assessing stereo vision by using a stereo graphic representation of a mobility course that measures the virtual distance of a user to the virtual objects and can be used to measure depth perception and the user’s sense of proximity to objects. The tester can query how many steps does the user need to take to get to the door or to a stop sign, for example.

In some embodiments, a virtual mobility test or a related test presentation may be generated or modified for individuals with additional (e.g., non-visual) conditions or disabilities. For example, for individuals with impaired mobility, instead of using their legs to walk, their hands can be used to point and/or click in the direction the individual chooses to move. In some examples, as a user is “moving” through a virtual mobility course, the user can point and click at various obstacles, thereby indicating that the individual recognizes them and is avoiding them. In another example, if movement of the legs or hands of a user cannot be monitored, VMTS 100 or related entities may utilize pupil tracking software to allow scoring based on the changes in direction of the gaze of the user. In some examples, data derived from pupil tracking software can complement data obtained from trackers tracking other body parts of the user. In some examples, when administering a virtual mobility test, VMTS 100 or related entities may provide auditory or tactile feedback to users with certain conditions for indicating whether or not they collided with an object. In such examples, auditory feedback may be provided through earphones or speakers on a headset and tactile feedback may be provided using vibrations via sensors 110 (e.g., on the feet, hands or headset depending on the location of the obstacle).

In some embodiments, VMTS 100 or one or more modules therein may utilize tracking of various user related metrics during a virtual mobility test and may use these metrics along with visual response data when analyzing the user performance and/or determining a related score. Such metrics may include: heart rate metrics, eye tracking metrics, respiratory tracking metrics, neurologic metrics (e.g., what part of the brain is excited and where, when; through electroencephalogram (EEG) sensors), auditory response metrics (e.g., to determine how those relate to visual performance since individuals with visual deficits may have enhanced auditory responses); distance sensitivity metrics (e.g., using LIDAR to measure a user-perceived distance to an object). In some embodiments, VMTS 100 or one or more modules therein may utilize pupillary light reflexes (e.g., captured during pupil tracking) for providing additional information regarding consensual response (and the function of sensorineural pathways leading to this response) as well as emotional responses and sympathetic tone.

In some embodiments, a virtual mobility test or a related test presentation may be generated or modified for data capturing or related analyses. For example, VMTS 100 or one or more modules therein may administer a test to normally-sighted control individuals then may administer the test one or more subsequent times under conditions where the disease is mimicked through a user’s display (e.g., VR googles).

In some examples, a control population of normally-sighted individuals is used to compare responses with a set of individuals with a form of RP that can result in decreased light sensitivity, blurring of vision, and visual field defects. In some examples, the virtual mobility test that is given to both normally-sighted individuals and those with RP may be the same or similar to the test described in Appendix A. After the virtual mobility test has been administered to the normally-sighted individuals, those individuals may be given another set of tests where the symptoms of RP are mimicked in presentation of the scene (e.g., via VR goggles). In some example, mimicking the symptoms in presentation may include significantly reducing the lighting in the virtual scene, blurring (e.g., Gaussian blurring) central vision in the virtual scene, blacking out or blurring patches of the peripheral vision fields in the virtual scene. In such examples, the performance of the individuals tested under conditions mimicking this disorder may be measured. The data under these conditions can be used as either a “control” group for virtual mobility performance or to control for the validity of the test.

In some examples, a control population of normally-sighted individuals is used to compare responses with a set of individuals with Stargardt disease that can result in poor visual acuity and poor color discrimination. In some examples, the virtual mobility test that is given to both normally-sighted individuals and those with Stargardt disease may incorporates a path defined by very large arrows and obstacles with colors that differ only slightly from the color of the background or colors used in the test may be in greyscale. After the virtual mobility test has been administered to the normally-sighted individuals, those individuals may be given another set of tests where the symptoms of Stargardt disease are mimicked in presentation of a virtual scene (e.g., via VR goggles). In some example, mimicking the symptoms in presentation may include blurring (e.g., Gaussian blurring) central vision when displaying the virtual scene. As such, the test may be easy for the normally-sighted individuals until their second round of testing. In such examples, the performance of the individuals tested under conditions mimicking this disorder may be measured. The data under these conditions can be used as either a “control” group for virtual mobility performance or to control for the validity of the test.

In some embodiments, conditions mimicking a vision condition or a related state can be inserted randomly or at defined moments while testing normally-sighted individuals. In some embodiments, a specific vision loss of a given patient could be emulated in a control patient. For example, data relating to visual fields, visual acuity, color vision, etc. that is measured in the clinic for a given user can be used to mimic this condition in the goggles for another user.

In some embodiments, various modifications of a virtual mobility test or a related scene presentation may be performed in order to mimic various visual conditions, including, for example, turning colors to greyscale, eliminating a specific color, presenting test objects only in a single color, showing gradient shading across objects or show mono-color shading (e.g., as described in Appendix A), rendering meshes, showing edges of objects only, inverting or reversing images, rotating images, hiding shadows, or distorting perspective (e.g., making things appear closer or farther).

In some embodiments, VMTS 100 or one or more modules therein may utilize virtual mobility test or related presentations for education purposes. For example, VMTS 100 or one or more modules therein may generate and administer a virtual mobility test that mimics a given vision condition. In this example, mimicking a vision condition may involve affecting a presentation of a virtual mobility course, e.g., blurring and/or dimming a virtual scene shown in a VR headset so that a user with normal sight (e.g., 20/20 vision and no known vision conditions) experiences symptoms of the vision condition.

In some examples, a virtual mobility test that mimics a given vision condition may be given to caregivers, medical students, family members, social workers, policy makers, insurance providers, architects, educational testing administrators, traffic controllers, etc., thereby providing. first-hand experience regarding the daily challenges faced by individuals with vision conditions. By experiencing visual disabilities in this manner, those individuals can better design living and working conditions for enhancing the safety and visual experience of those with various vision impairments.

In some embodiments, VMTS 100 or one or more modules therein may provide “light” version of a particular virtual mobility test and/or may utilize available technology for presenting the test. For example, a “light” version of a particular VR-based virtual mobility test may be generated or adapted for an augmented reality (AR) experience on a smartphone or a tablet computer, e.g., when VR testing is not feasible or easily accessible. AR-based testing could assist remote or underserved populations or those that are isolated due to disease or economic factors. Such AR-based testing may be used in conjunction with telemedicine or virtual education.

In some embodiments, VMTS 100 or one or more modules therein may utilize various technologies, e.g., artificial intelligence and/or AR, for diagnostics and training. For example, the ability of some AR headsets to see the room as well as a virtual scene simultaneously (also referred to here as “inside outside viewing”) may be usable for incorporating a user’s real-world home life (or work-life) into a course that allows the user to practice and improve their navigation. In this example, AR-based courses can be useful for training individuals to better use their (poor) vision. In some examples, AR-based testing may be useful for in-home monitoring of a user’s current condition and/or progress. In such examples, by using AR-based testing and/or portable and easy-to-use hardware, the user’s vision function can still be monitored even in less than ideal environments or situations, such as pandemics. In some examples, using AI based algorithms and/or associated metrics, VMTS 100 or one or more modules therein may gather additional data to identify trends in user performance and also to train or improve the ability of the user to better use their impaired vision, e.g., by measuring or identifying progress. In such embodiments, using AI based algorithms and/or associated metrics, VMTS 100 or one or more modules therein may identify and improve aspects of a test or related presentation for various goals, e.g., improving diagnostics and training efficiency.

In some embodiments, VMTS 100 or one or more modules therein may allow a multi-user mode or social engagement aspect to virtual mobility testing. For example, VMTS 100 or one or more modules therein may administer a virtual mobility test to multiple users concurrently, where the users can interact and related interaction (or avoidance of collisions) can be measured and/or evaluated.

It will also be appreciated that the above described modules, components, and nodes in FIG. 1 are for illustrative purposes and that features or portions of features described herein may be performed by different and/or additional modules, components, or nodes than those depicted in FIG. 1. It will also be appreciated that some modules and/or components may be combined and/or integrated. For example, user display 108 and processing platform 101 may be integrated into a single computing device, module, or system. For example, a VIVE VR system, a mobile computing device, or smartphone configured with appropriate VR software, hardware, and mobility testing logic may generate a virtual environment and may perform and analyze a mobility test in the virtual environment. In this example, the mobile computing device or smartphone may also display and record a user’s progress through the virtual mobility test.

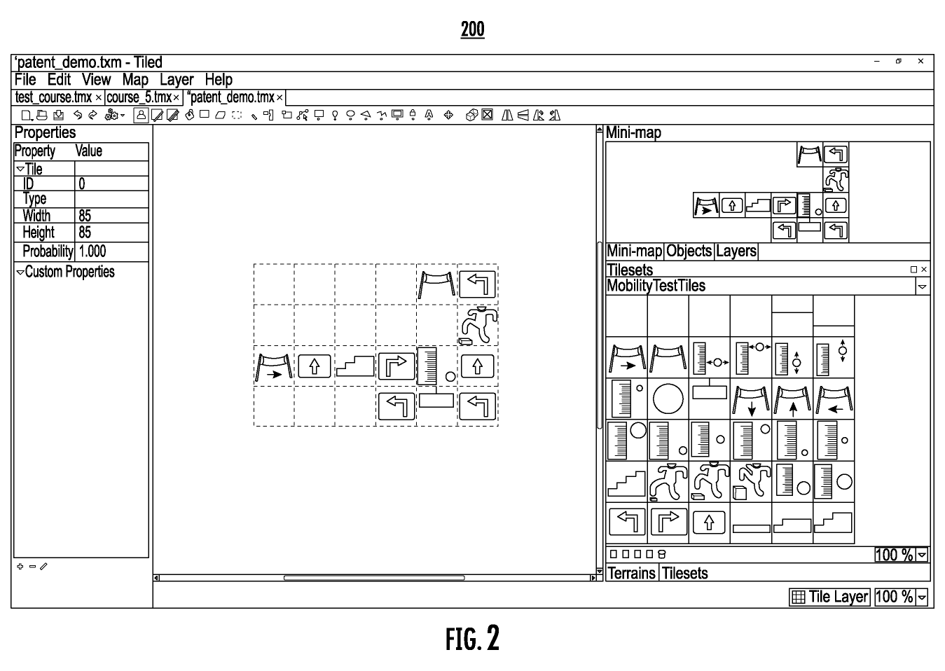

FIG. 2 is a diagram 200 illustrating an example template for a virtual mobility test. In some embodiments, a virtual environment and/or a related obstacle course may utilize a template generated using a program called Tiled Map Editor (http://www.mapeditor.org). In such embodiments, a user may select the File Menu, select the New Option, and then select the New Map Option (File Menu->New->New Map) to generate a new map. In some embodiments, the user may configurable various aspects of the new map, e.g., the new map may be set to ‘orthogonal’ orientation, the tile format may be set to ‘CSV’, the tile render order may be set to ‘Left Up’.

In some embodiments, the tile size may be a width of 85 pixels (px) and a height of 85 px. The Map size may be fixed, and the number of tiles may be user-configurable. In some embodiments, the dimensions may be set to a width of 5 tiles and a length of 10 tiles (e.g., 5 ft X 10 ft).

In some embodiments, the name of the map may be selected or provided when the map is saved (e.g., File Menu->Save As).

In some embodiments, a tile set may be added to the template (File Menu->New->New Tile set). A tile set may include a number of tile types, e.g., basic path tiles are straight, turn left, and turn right. A tile set may also include variations of a tile type, e.g., a straight tile type may include a hanging obstacle tiles, button tiles, and step over tiles. In some embodiments, a tile set may also provide one or more colors, images, and/or textures for the path or tiles in a template. In some embodiments, a tile set may be named and a browse button may be used to select an image file source and appropriate tile width and height may also be inputted (e.g., 85 px for tile width and height).

In some embodiments, to place a tile on the map, select or click a tile from your tile set and then click on the square on which to place the selected tile. For example, to create a standard mobility test or related obstacle course, a continuous path may be created from one of the start tiles to the finish tile.

In some embodiments, after a template is created, the template may be exported and saved to a location for use by VMTS 100 or other entities (File Menu->Export As). For example, after exporting a template as a file name map.csv, the file may be stored in a folder along with a config.csv containing additional configuration information associated with a virtual mobility test. In this example, VMTS 100 or related entities may use the CSV files to generate a virtual mobility test.

In some embodiments, a configuration file (e.g., config.csv) may be used to add or remove courses and/or configure courses used in a virtual mobility test. Example configuration settings for a virtual mobility test are listed below: play_area_width and play_area_height

tile _length The value is the desired length and width of each tile in meters. swings_per_sec The value is indicates the number of seconds it takes for a swinging obstacle to make its full range of motion. subject_height The value is the height of the user (test participant) in meters. (Some values in the configuration file may be a fraction of the user’s height. Changing this value may affect hanging_obstacle_height, arrow_height, low_height, med_height, high_height, med_obstacle_radius, and big_obstacle_radius.) hanging_obstacle_height The value is the distance between the floor and the bottom of the obstacle. (The value may be a fraction of the height of the user.) arrow_height The value is the distance between the guiding arrows and the floor. (The value may be a fraction of the height of the user.) low_height The value is the distance between the center of low floating obstacles and the floor and height of low box obstacles. (The value may be a fraction of the height of the user.) med_height The value is the distance between the center of medium floating obstacles and the floor and height of high box obstacles. (The value may be a fraction of the height of the user.) high_height

small_obstacle_radius The value is the radius of small floating obstacles. (The value may be a fraction of the height of the user.) med_obstacle_radius The value is the radius of medium floating obstacles. (The value may be a fraction of the height of the user.) big_obstacle_radius The value is the radius of large floating obstacles. (The value may be a fraction of the height of the user.) tiny_step The value is the height of very small step-over obstacles. (The value may be a fraction of the height of the user.) small_step The value is the height of small step-over obstacles. (The value may be a fraction of the height of the user.) big_step The value is the height of big step-over obstacles. (The value may be a fraction of the height of the user.) huge_step The value is the height of very big step-over obstacles. (The value may be a fraction of the height of the user.) box_length The value is the width and depth of box obstacles. (The value may be a fraction of tile length.) parking meter The height is 5 feet with the shape of a parking meter. open dishwasher door The door of a box-like dishwasher may be open anywhere between a 5-90 degree angle and jut into the pathway. arrow_local_scale

arrow_luminance The value indicates the luminance of guiding arrows in the scene (the virtual environment or course). The luminance may be measured in lumens (lux) and the maximum value may be operator-configurable or may be user display dependent. button_luminance The value indicates the luminance of all buttons in the scene (the virtual environment or course). The luminance may be measured in lux and the maximum may be operator-configurable or may be user display dependent. obstacle_luminance The value indicates the luminance of all obstacles (e.g., box-shaped, floating, swinging, or hanging obstacles) in the scene. The luminance may be measured in lux and the maximum value may be operator-configurable or may be user display dependent. foot_luminance The value indicates the luminance of the user’s hands and feet in the scene. The luminance may be measured in lux and the maximum value may be operator-configurable or may be user display dependent. finish_line_luminance The value indicates the luminance of the finish line in the scene. The luminance may be measured in lux and the maximum value may be operator-configurable or may be user display dependent. num_courses The value indicates the number of file names referenced in this configuration file. Whatever this value is, there should be that many file names ending in .csv following it, and each one of those file names should correspond to a .csv file that is in the same folder as the configuration file.

It will also be appreciated that the above described files and data in FIG. 2 are for illustrative purposes and that VMTS 100 or related entities may use additional and/or different files and data than those depicted in FIG. 2.

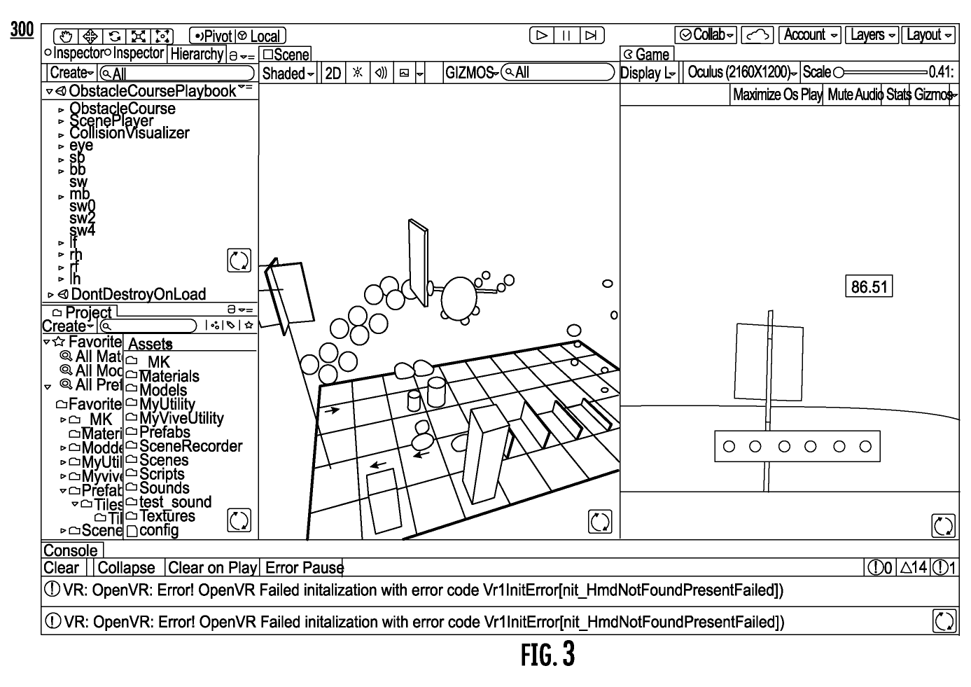

FIG. 3 is a diagram 300 illustrating a user performing a virtual mobility test. In FIG. 3, a test observer’s view is shown on the left panel and a test user’s view is shown on the right panel. Referring to FIG. 3, the test user may be at the termination point of the course looking in the direction of a green arrow. In the VR scene, a test user’s head, eyes, hands and feet may appear white. The test observer’s view may be capable of displaying various views (e.g., first person view, overhead (bird’s eye) view, or a third person view) of a related obstacle course and may be adjustable on-the-fly. The test user’s view may also be capable of displaying various views and may be adjustable on-the-fly, but may default to the first-person view. The termination point may be indicated by black flag and the test user may mark the completion of the course by touching the flag with his/her favored hand. Alternatively, the test user may walk into the flag. On the right panel, the user’s “touch” may be indicated with a red sphere.

It will be appreciated that FIG. 3 is for illustrative purposes and that various virtual mobility tests may include additional and/or different features than those depicted in FIG. 3.

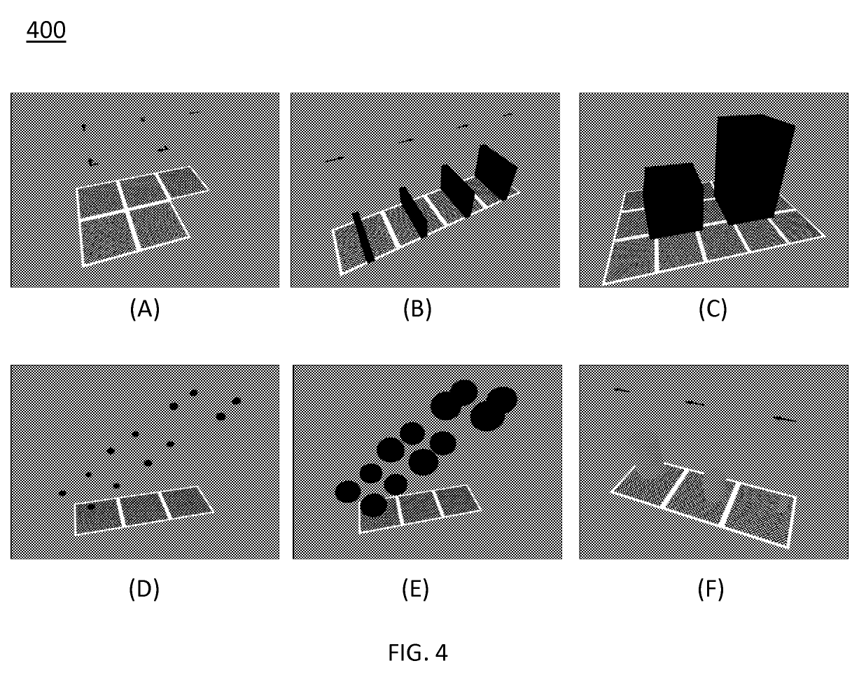

FIG. 4 is a diagram 400 illustrating example objects in a virtual mobility test. Referring to FIG. 4, images A-F depict example objects that may include in a virtual mobility test. Image A depicts tiles and arrows showing the path (e.g., pointing forward, to the left, or to the right). In some embodiments, arrows may be depicted on the tiles (and not floating above the tiles). Image B depicts step-over obstacles and arrows showing the required direction of movement. Image C depicts box-shaped obstacles. Image D depicts small floating obstacles (e.g., a group of 12) at different levels of a user’s body (e.g., from ankle to head height). Image E depicts large floating obstacles (e.g., a group of 10) at different levels of a user’s body (e.g., from ankle to head height). Image F depicts obstacles that a user must step on (e.g., to mimic stairs, rocks, etc.). These obstacles may depress (e.g., sink into the floor or tiles) as the user steps on them. In some embodiments, arrows may be depicted on the tiles (and not floating above or adjacent to the tiles).

It will be appreciated that FIG. 4 is for illustrative purposes and that other objects may be used in a virtual mobility test than those depicted in FIG. 4. For example, a virtual mobility test may include an obstacle course containing small, medium, and large floating obstacles, parking meter-shaped posts, or doors (e.g., partially open dishwasher doors) or gates that jut into the path.

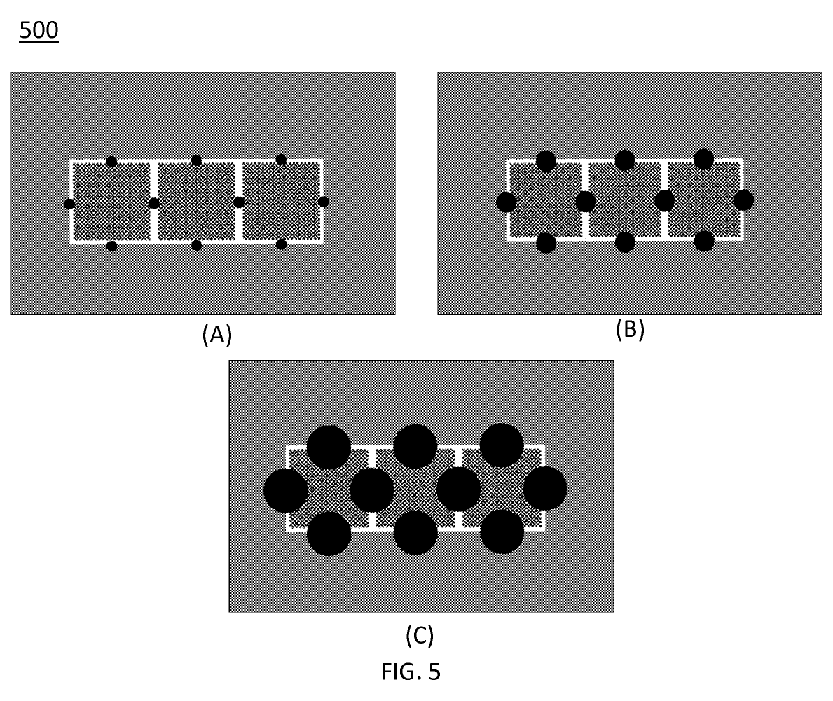

FIG. 5 is a diagram 500 illustrating various sized obstacles in a virtual environment. In some embodiments, a virtual mobility test or related objects therein may be adjustable. For example, VMTS 100 or one or modules therein may scale or resize obstacles based on a test user’s height or other physical characteristics. In this example, scalable obstacle courses may be useful for comparisons of performance of individuals who differ in height as the user’s height (e.g., distance of the eyes to the objects on the ground) affects visual resolution (e.g., visual acuity). The ability to resize objects in a virtual mobility test is also useful for following the visual performance of a child over time, e.g., as the child will grow and become an adult. In some embodiments, scaling an obstacle course may also be useful to ensure that obstacles or elements in the virtual environment (e.g., tiles that make of a course segments) are sized appropriately (e.g., so that a user’s foot can fit along an approved path through the virtual obstacle course).

Referring to FIG. 5, images A-C depict different size obstacles from an overhead view. For example, image A depicts an overhead view of small floating obstacles, image B depicts an overhead view of medium floating obstacles, and image C depicts an overhead view of large floating obstacles.

It will be appreciated that FIG. 5 is for illustrative purposes and that objects may be scaled in more precise terms and/or with more granularity (e.g., a percentage or fraction of a test user’s height). For example, a virtual mobility test may include an obstacle course containing obstacles that appear to be 18.76% of the height of a test user.

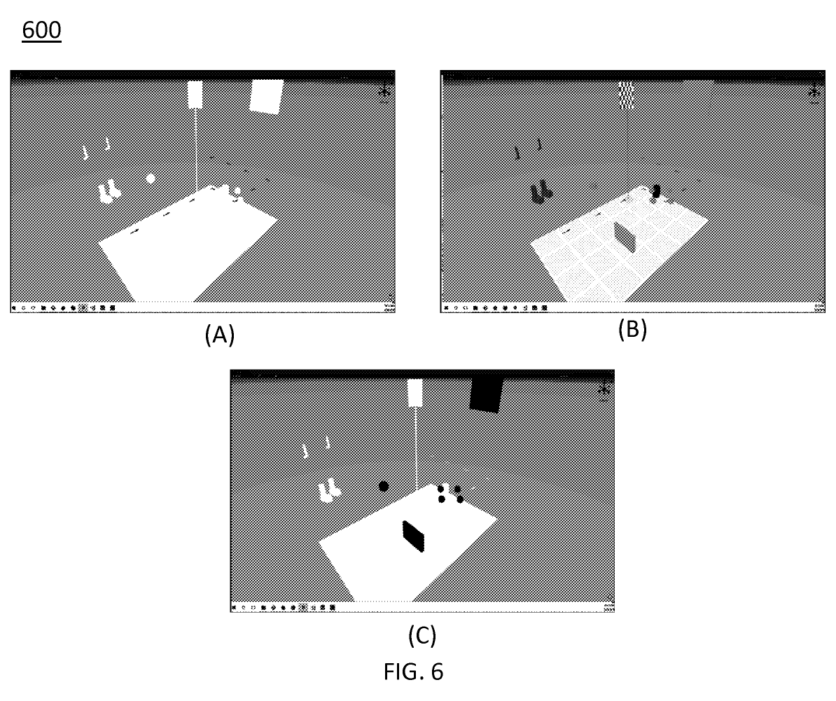

FIG. 6 is a diagram 600 illustrating virtual mobility tests with various lighting conditions. In some embodiments, lighting conditions in a virtual mobility test may be adjustable. For example, VMTS 100 or one or more modules therein may adjust lighting conditions for a virtual environment or related obstacle course associated with a virtual mobility test. For example, VMTS 100 or one or more modules therein may adjust luminance of various objects (e.g., obstacles, path arrows, hands, head, and feet, finish line, and/or floor tiles) associated with a virtual mobility test.

In some embodiments, individual obstacles and/or groups of obstacles can be assigned different luminance, contrast, shading, outlines, and/or color. In some embodiments, each condition or setting may be assigned a relative value or an absolute value. For example, assuming luminance can be from 0.1 lux to 400 lux, a first obstacle can be displayed at 50 lux and a second obstacle can be assigned to a percentage of the first obstacle (e.g., 70% or 35 lux). In this example, regardless of a luminance value, some objects in a virtual mobility test may have a fixed luminance (e.g., a finish flag).

Referring to FIG. 6, images A-C depict a mobility test under different luminance conditions with arrows highlighted for illustrative purposes. For example, image A shows a mobility test displayed under low luminance conditions (e.g., about 1 lux); image B shows a mobility test with a step obstacle displayed under medium luminance conditions (e.g., about 100 lux); and image C shows a mobility test with a step obstacle and other objects displayed under high luminance conditions (e.g., about 400 lux).

It will be appreciated that FIG. 6 is for illustrative purposes and that different and/or additional aspects of the virtual mobility test than those depicted in FIG. 6.

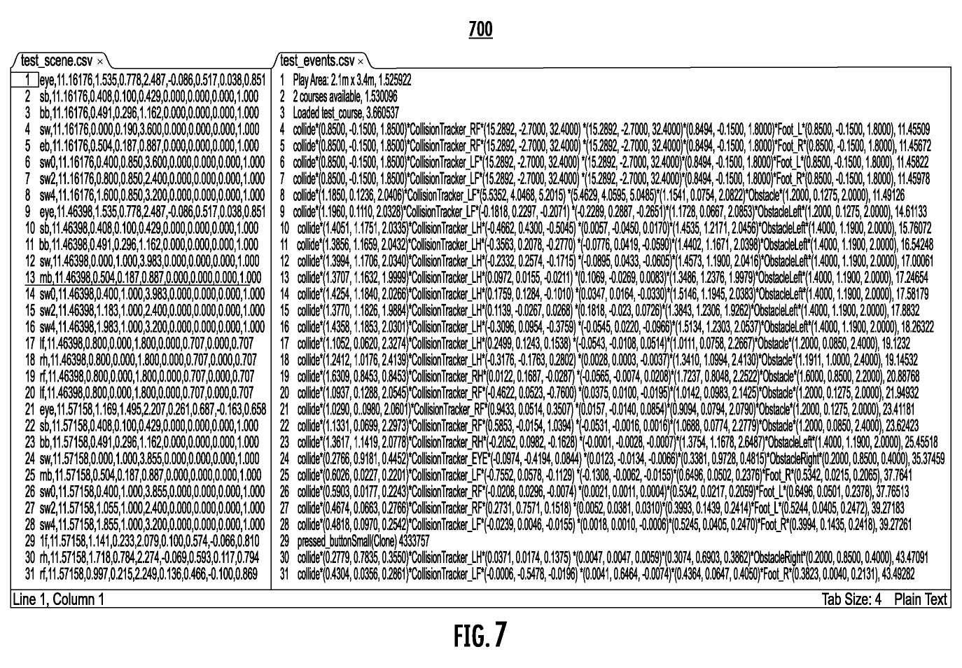

FIG. 7 is a diagram 700 illustrating example data captured during a virtual mobility test. In some embodiments, VMTS 100 or one or more modules therein (e.g., TC 102 and/or sensor data collector 104) may analyze various data associated with a virtual mobility test. For example, VMTS 100 or modules therein may gather data from sensors 110, information regarding the virtual environment (e.g., locations and sizes of obstacles, path, etc.), and/or one or more video cameras. In this example, the data captured may be measured and analyzed using quantitative analysis (e.g., based on objective criteria).

Referring to FIG. 7, captured data may be stored in one or more files (e.g., test_events.csv and test_scene.csv files). Example captured data may include details of an obstacle course in a virtual mobility test (e.g., play area, etc.), a particular configuration of the course, a height of a test user, sensor locations (e.g., head, hands, feet) as a function of time (e.g., indicative of body movements), direction of gaze, acceleration and deceleration, leaning over to look more closely at an object, and/or amount of time interacting with each obstacle.

It will be appreciated that FIG. 7 is for illustrative purposes and that different and/or additional data than those depicted in FIG. 7 may be captured or obtained during a virtual mobility test.

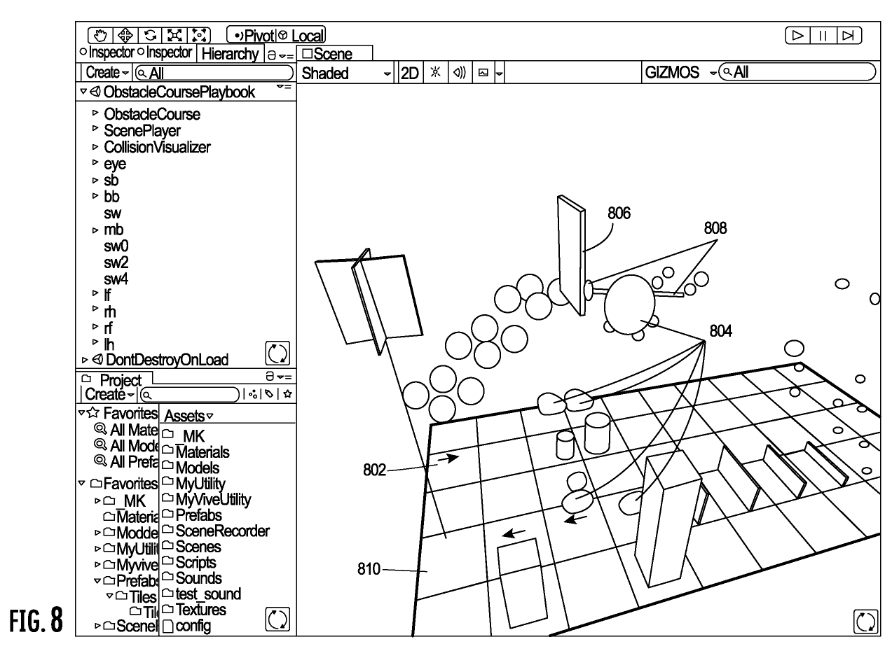

FIG. 8 is a diagram 800 illustrating various aspects of an example virtual mobility test. In some embodiments, VMTS 100 or one or more modules therein may be capable of providing real-time or near real-time playback of a user’s performance during a virtual mobility test. In some embodiments, VMTS 100 or one or more modules therein may be capable of recording a user’s performance during a virtual mobility test. For example, VMTS 100 or modules therein may use gathered data from sensors 110, and/or other input, to create an avatar representing the user in the virtual mobility test and may depict the avatar interacting with various objects in the virtual mobility test. For example, a video or playback of a user performing a virtual mobility test may depict the user’s head, eyes, hands, and feet appear white on the video and can depict the user walking through an obstacle course toward the termination point (e.g., a finish flag) of the course. In this example, to emphasize the user bumping into the hanging sign and stepping backwards, a green arrow may point to a red circle located at the location of the collision.

Referring to FIG. 8, a snapshot from a playback of a user performing a virtual mobility test is shown. In the snapshot, start location 802 represents the start of an obstacle course; avatar 804 represents the user’s head, hands, and feet; floating obstacle 806 represents a head height obstacle in the obstacle course (e.g., one way to avoid such an obstacle is to duck); indicators 808 represent a collision circle indicating where a collision between the user and the virtual environment occurred and an arrow pointing to the collision circle; and finish location 810 represents the end of the obstacle course. It will be appreciated that FIG. 8 is for illustrative purposes and that different and/or additional aspects than those depicted in FIG. 8 may be part of a virtual mobility test.

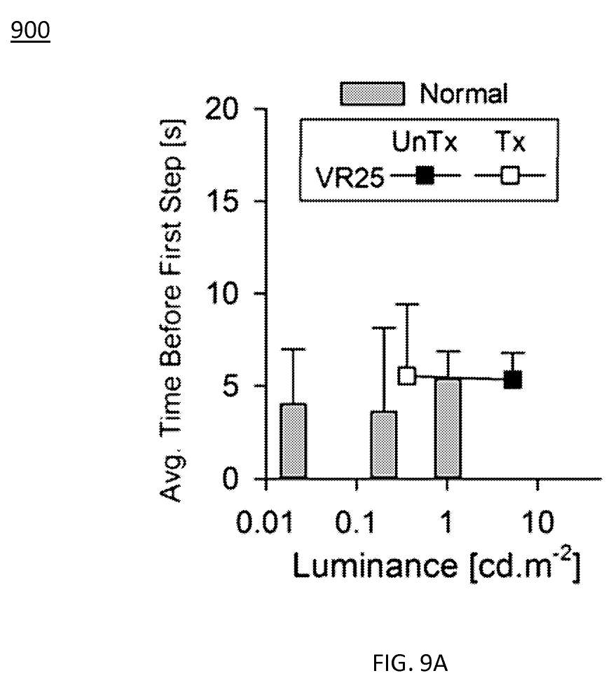

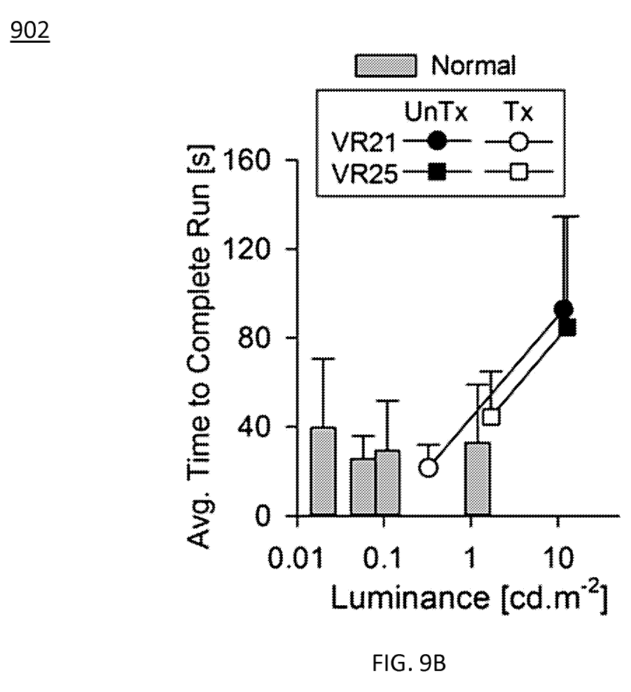

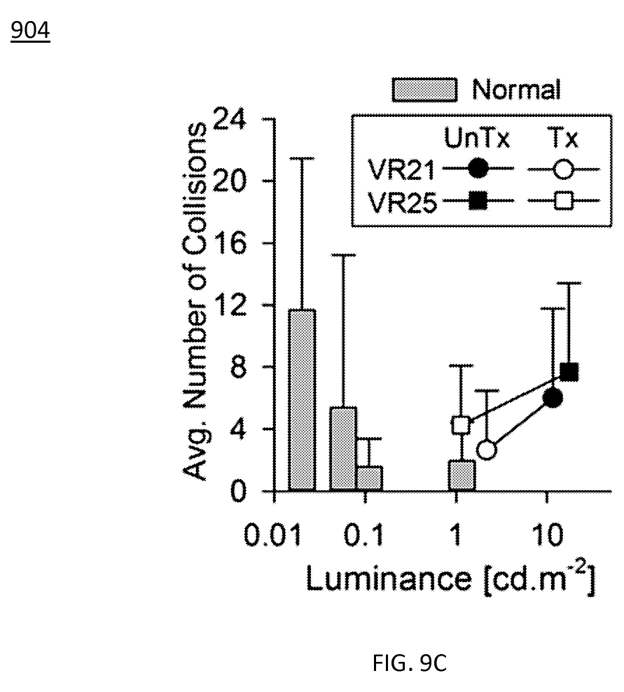

FIGS. 9A-9C depict graphs indicating various data gathered from subjects in a study using virtual mobility testing. The study evaluated seven normally sighted control subjects and three subjects with RPE65-associated Leber Congenital Amaurosis. Moreover, during the study, two of the three RPE65-associated subjects were given gene therapy for treating their visual dysfunction. To gather subject related data, subjects in the study underwent perimetry and full-field sensitivity testing along with testing involving an VTMS implementation. Additional details regarding the study and aspects of the VTMS implementation are found in Appendix A. The disclosures of Appendix A and the references listed therein are each incorporated herein by reference in its entirety to the extent not inconsistent herewith and to the extent that it supplements, explains, provides a background for, or teaches methods, techniques, and/or systems employed herein and is hereby incorporated by reference in its entirety.

Referring to FIG. 9A, a graph 900 may be a box plot indicating average time before first step[s] in an “arrows only” virtual mobility test for subjects in the study of Appendix A. Graph 900 shows average time before first step[s] in the virtual mobility tests in relation to the luminance (e.g., an average brightness) associated with the tests. Further, graph 900 provides data for comparing control or “normal” subjects in this study to an RPE65-associated subject ‘VR25’ that undergoes gene therapy. Data points for subject ‘VR25’ before treatment (pre-Tx = black symbols) are connected to post-treatment (post-Tx; white symbols) values to demonstrate change in performance (e.g., improvements or effects related to gene therapy).

Referring to FIG. 9B, a graph 902 may be a box plot indicating average number of collisions in an “arrows and obstacles” virtual mobility test for subjects in the study of Appendix A. Graph 902 also shows average number of collisions in the virtual mobility tests in relation to the luminance (e.g., an average brightness) associated with the tests. Further, graph 902 provides data for comparing control or “normal” subjects in this study to RPE65-associated subjects ‘VR21’ and ‘VR25’ that undergo gene therapy. Data points for subjects ‘VR21’ and ‘VR25’ before treatment (pre-Tx = black symbols) are connected to post-treatment (post-Tx; white symbols) values to demonstrate change in performance (e.g., improvements or effects related to gene therapy).

Referring to FIG. 9C, a graph 904 may be a box plot indicating average times to complete run[s] (e.g., of the test course) in an “arrows and obstacles” virtual mobility test for subjects in the study of Appendix A. Graph 904 also shows average times to complete run[s] in the virtual mobility tests in relation to the luminance (e.g., an average brightness) associated with the tests. Further, graph 904 provides data for comparing control or “normal” subjects in this study to RPE65-associated subjects ‘VR21’ and ‘VR25’ that undergo gene therapy. Data points for subjects ‘VR21’ and ‘VR25’ before treatment (pre-Tx = black symbols) are connected to post-treatment (post-Tx; white symbols) values to demonstrate change in performance (e.g., improvements or effects related to gene therapy).

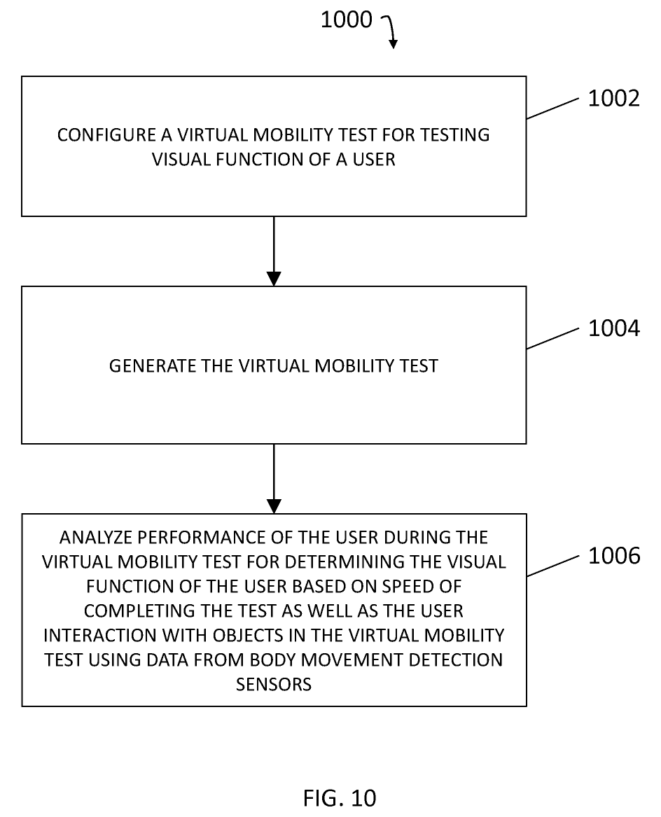

FIG. 10 is a flow chart illustrating an example process 1000 for testing visual function using a virtual environment. In some embodiments, example process 1000 described herein, or portions thereof, may be performed at or performed by VMTS 100, processing platform 101, TC 102, sensor data collector 104, user display 108, and/or another module or node. Referring to example process 1000, in step 1002, a virtual mobility test may be configured for testing visual function of a user. For example, VMTS 100 may use configuration files containing settings and/or configuration information for configuring a virtual mobility test or a related obstacle course. In some embodiments, configuring a virtual mobility test may include configuring the virtual mobility test based on the user, e.g., physical characteristics or a vision condition (e.g., an eye disease or other condition that effects vision).

In step 1004, the virtual mobility test may be generated. For example, VMTS 100 may generate and display a virtual mobility test to user display 108.

In step 1006, performance of the user during the virtual mobility test may be analyzed for determining the visual function of the user based on user interaction with objects in the virtual mobility test using data obtained from body movement detection sensors. For example, VMTS 100 or related entities may receive data collected from sensors 110 to determine whether a user collided with an obstacle in a virtual mobility test. In this example, the number or amounts of collisions may affect a generated score indicating performance of the user regarding the virtual mobility test. In some embodiments, configuring a virtual mobility test may include configuring the virtual mobility test to test a right eye, a left eye, or both eyes. In some embodiments, configuring a virtual mobility test may include configuring luminance, shadow, color, contrast, gradients of contrast/color on the surface of the obstacles or enhanced contrast, “reflectance” or color of borders of obstacles or a lighting condition associated with one or more of the objects in the virtual mobility test based on configuration information.

In some embodiments, analyzing performance of a user may include evaluating the effectiveness of a gene therapy or related treatment for a vision condition of the user. For example, VMTS 100 or related entities may administer and use data collected from one or more virtual mobility tests involving a user undergoing or that has undergone gene therapy. In this example, VMTS 100 or related entities may evaluate performance from collected test results or related metrics and may correlate this to the gene therapy, related treatments, and/or other factors. Continuing with this example, VMTS 100 or related entities may use this evaluation to identify and report on the effectiveness of the gene therapy, e.g., by indicating the amount or extent of progress or improvement the user has shown since the start of the gene therapy.

In some embodiments, configuring a virtual mobility test may include configuring the height of one or more of the objects in the virtual mobility test and/or configuring the size of one or more of the objects in the virtual mobility test based on configuration information.

In some embodiments, configuration information may include the height or other attributes of the user, condition-based information (e.g., aspects of cognition, perception, eye or vision condition, etc.), user-inputted information, operator-inputted information, or dynamic information.

In some embodiments, generating a virtual mobility test may include providing auditory or haptic feedback to the user when a feedback condition occurs, wherein the feedback condition may include a collision between the user and one or more of the objects in the virtual mobility test, the user leaves a designated path or course associated with the virtual mobility test, or a predetermined amount of progress has not occurred in a predetermined amount of time.

In some embodiments, generating a virtual mobility test may include capturing the data from body movement detection sensors and using the data to output a video of the user’s progress through the virtual mobility test. For example, a video of a user’s progress through the virtual mobility test may include an avatar representing the user and/or their body movements. In some embodiments, objects in a virtual mobility test may include a tile, an obstacle, a box obstacle, a step-over obstacle, a hanging or swinging obstacle, a floating obstacle, a starting line, a finish line, a finish flag, a guide arrow, or a button obstacle.

In some embodiments, testing may be done in a multi-step fashion in order to isolate the role of central vision versus peripheral vision. For example, a virtual mobility test or a related test may be configured to initially identify a luminance threshold value for the user to identify colored (red, for example) arrows on the path. This luminance threshold value may then be held constant in subsequent tests for central vision while luminance of the obstacles is modified in order to elicit the sensitivity of the user’s peripheral vision.

It will be appreciated that process 1000 is for illustrative purposes and that different and/or additional actions may be used. It will also be appreciated that various actions described herein may occur in a different order or sequence.

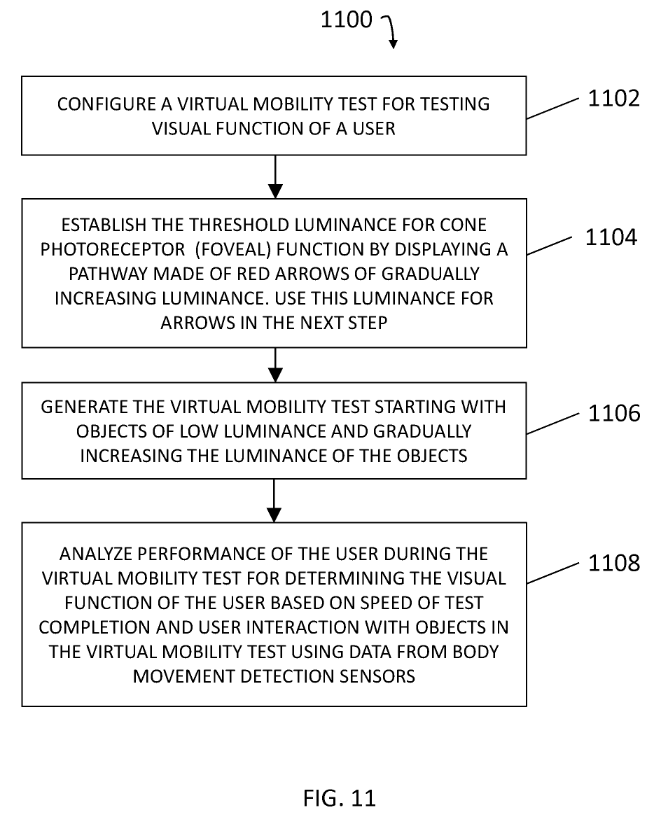

FIG. 11 is a flow chart illustrating an example process 1100 for dissecting or analyzing two different parameters of visual function. In some embodiments, example process 1100 described herein, or portions thereof, may be performed at or performed by VMTS 100, processing platform 101, TC 102, sensor data collector 104, user display 108, and/or another module or node.

Referring to example process 1100, in step 1102, a virtual mobility test may be configured for testing visual function of a user. For example, VMTS 100 may use configuration files containing settings and/or configuration information for configuring a virtual mobility test or a related obstacle course.

In some embodiments, configuring a virtual mobility test may include configuring the virtual mobility test based on the user, e.g., physical characteristics or a vision condition (e.g., an eye disease or other condition that effects vision).

In step 1104, the threshold luminance for cone photoreceptor (e.g., foveal or center of vision) function of the user may be established or determined. For example, VMTS 100 may generate and display a pathway of red (or other color) arrows, where the arrows are gradually increasing in luminance.

In step 1106, using the threshold luminance established in step 1104, a virtual mobility test may be generated. For example, VMTS 100 may generate and display a virtual mobility test to user display 108, where the objects (e.g., obstacles) at the start of the virtual mobility test are of a low luminance (e.g., as determined by a standard or based on the user’s established threshold luminance from step 1104). In this example, as the user moves through the virtual mobility test the encountered objects may gradually increase in luminance.

In step 1108, performance of the user during the virtual mobility test may be analyzed for determining the visual function of the user based on speed of test completion and user interaction with objects in the virtual mobility test using data obtained from body movement detection sensors. For example, VMTS 100 or related entities may receive data collected from sensors 110 to determine whether a user collided with an obstacle in a virtual mobility test. In this example, the number or amounts of collisions may affect a generated score indicating performance of the user regarding the virtual mobility test.

In some embodiments, analyzing performance of a user may include analyzing sensor data using detection threshold values and/or related formulas to ignore or mitigate sensor related issues, e.g., false positives for collision events. For example, if a user tries to step over the “stepover” obstacle but strikes the obstacle in the process, if sensor data from feet trackers indicates that each leg lift distance meets or exceeds a “stepover” threshold value then a related stepover collision event may be voided or ignored by VMTS 100 or related entities. In another example, if a user attempts to duck below a hanging obstacle by dropping his/her head a few inches but grazes the hanging obstacle in the process, VMTS 100 or related entities may void or ignore a related collision event if sensor data from a head tracker meets or exceeds a “duck” threshold value.

In some embodiments, analyzing performance of a user may include evaluating the effectiveness of a gene therapy or related treatment for a vision condition of the user. For example, VMTS 100 or related entities may administer and use data collected from one or more virtual mobility tests involving a user undergoing or that has undergone gene therapy. In this example, VMTS 100 or related entities may evaluate performance from collected test results or related metrics and may correlate this to the gene therapy, related treatments, and/or other factors. Continuing with this example, VMTS 100 or related entities may use this evaluation to identify and report on the effectiveness of the gene therapy, e.g., by indicating the amount or extent of progress or improvement the user has shown since the start of the gene therapy.

In some embodiments, analyzing performance of a user may include measuring one or more symptoms of a vision condition or diagnosing a user with a vision condition. For example, VMTS 100 or related entities may administer and use data collected from one or more virtual mobility tests involving presenting a virtual mobility course under different levels of luminance. In this example, VMTS 100 or related entities may evaluate performance from collected test results or related metrics and may correlate this information. Continuing with this example, VMTS 100 or related entities may use this information to diagnose forms of RP or Leber’s congenital amaurosis and/or to measure related symptoms.

It will be appreciated that process 1100 is for illustrative purposes and that different and/or additional actions may be used. It will also be appreciated that various actions described herein may occur in a different order or sequence.

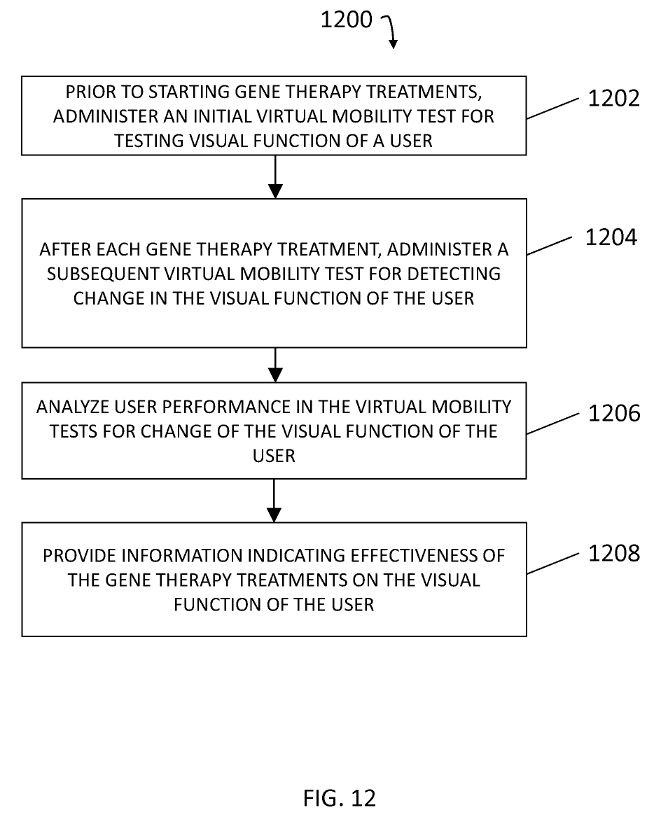

FIG. 12 is a flow chart illustrating an example process 1200 for evaluating effectiveness of gene therapy on visual function of a user using virtual mobility tests. In some embodiments, example process 1200 described herein, or portions thereof, may be performed at or performed by VMTS 100, processing platform 101, TC 102, sensor data collector 104, user display 108, and/or another module or node.

In some embodiments, VMTS 100 may be usable to evaluate effectiveness of gene therapy for a user with a visual dysfunction. For example, a gene therapy (e.g., using voretigene neparvovec-rzyl via pars-plana vitrectomy and subretinal injection) may be usable for treating users that have vision impairment due to RPE65-associated Leber Congenital Amaurosis. In this example, VMTS 100 or related entities may administer and use data collected from one or more virtual mobility tests involving a user undergoing or that has undergone this gene therapy. In this example, VMTS 100 or related entities may evaluate performance from collected test results or related metrics and/or correlate this to the gene therapy, related treatments, and/or other factors. Continuing with this example, VMTS 100 or related entities may use this evaluation to identify and report on the effectiveness of the gene therapy, e.g., by indicating the amount or extent of progress or improvement the user has shown since the start of the gene therapy.

Referring to example process 1200, in step 1202, prior to starting gene therapy treatments, an initial virtual mobility test may be administered for testing visual function of a user. For example, VMTS 100 may use configuration files containing settings and/or configuration information for configuring a virtual mobility test or a related obstacle course. In this example, after configuration, the virtual mobility test may be administered to a user prior to gene therapy so as to establish a baseline for measuring change in visual function after gene therapy begins.

In step 1204, after each gene therapy treatment, a subsequent virtual mobility test may be administered for detecting a change in the visual function of the user. For example, a gene therapy treatment (e.g., using voretigene neparvovec-rzyl via pars-plana vitrectomy and subretinal injection) may be performed in a first eye of a user and then sometime later (e.g., two weeks later) another virtual mobility test may be administered for detecting a change in the visual function of the user, e.g., fewer collisions and/or faster course completion times relative to a baseline. In this example, after this virtual mobility test, another gene therapy treatment may be performed in a second eye of the user and then sometime later (e.g., two weeks later) a subsequent virtual mobility test may be administered for detecting a further change in the visual function of the user, e.g., fewer collisions and/or faster course completion times relative to the baseline and/or a prior virtual mobility test.

In some embodiments, virtual mobility tests may be adjusted or modified between test administrations to the same user so as to avoid or mitigate learning bias. For example, with physical mobility tests, the time required to modify a mobility test between administration may be prohibited. In this example, since the test remains the same, a user can learn a path for the mobility test and/or where obstacles are along the path and, therefore, test results may not accurately reflect the user’s true visual function or ability. In contrast to static physical mobility tests, VMTS 100 may adjust a path and/or location of obstacles of a virtual mobility test to prevent or mitigate learning bias. VMTS 100 may also utilize an algorithm for adjusting a virtual mobility test while still representing a particular skill level (e.g., the modified virtual mobility test is determined to be equivalent to an original virtual mobility test).

In step 1206, user performance in the virtual mobility tests may be analyzed for detecting the change of the visual function of the user. For example, metrics related to performance may be tracked for each test and any change (e.g., progress or improvement) may be identified. In this example, changes in performance or aspects therein may be measured using various techniques and/or formulas and may be represented in various forms, e.g., percentages, ratings, or scales.

In step 1208, information indicating effectiveness of the gene therapy treatments on the visual function of the user may be reported. For example, VMTS 100 or a related GUI may generate one or more visualizations (e.g., graphs 900-904) for visually depicting changes of visual function of a user in response to gene therapy treatments.

It will be appreciated that process 1200 is for illustrative purposes and that different and/or additional actions may be used. It will also be appreciated that various actions described herein may occur in a different order or sequence.

It should be noted that VMTS 100 and/or functionality described herein may constitute a special purpose computing device. Further, VMTS 100 and/or functionality described herein can improve the technological field of eye treatments and/or diagnosis. For example, by generating and using a virtual mobility test, a significant number of benefits can be achieved, including the ability to test visual function of a user quickly and easily without requiring expensive and time-consuming setup (e.g., extensive lighting requirements) needed for conventional mobility test. In this example, the virtual mobility test can also use data collected from sensors and the VR system to more effectively and more objectively analyze and/or score a user’s performance. The details provided here would also be applicable to augmented reality (AR) systems which could be delivered through glasses, thus facilitating usage.

It may be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A system comprising: at least one processor; and a memory, wherein the system is configured for: configuring a virtual mobility test for testing visual function of a user; generating the virtual mobility test; and analyzing performance of the user during the virtual mobility test for determining the visual function of the user based on user interaction with objects in the virtual mobility test using data from body movement detection sensors.

2. The system of claim 1 wherein configuring the virtual mobility test includes configuring the virtual mobility test based on the user or a vision condition; or wherein configuring the virtual mobility test includes configuring the virtual mobility test to test a right eye, a left eye, or both eyes.

3. The system of claim 1 wherein analyzing the performance of the user includes evaluating the effectiveness of a gene therapy or related treatment for a vision condition of the user, measuring one or more symptoms of a vision condition, or diagnosing the user with a vision condition.

4. The system of claim 1 wherein configuring the virtual mobility test includes configuring luminance, shadow, color, contrast, gradients of contrast or color on the surface of the objects, reflectance or color of borders of the objects, or a lighting condition associated with one or more of the objects in the virtual mobility test based on configuration information.

5. The system of claim 1 wherein configuring the virtual mobility test includes: configuring the height of one or more of the objects in the virtual mobility test and/or configuring the size of one or more of the objects in the virtual mobility test based on configuration information.

6. The system of claim 5 wherein the configuration information includes the height or other attributes of the user, condition-based information; user-inputted information, operator-inputted information, or dynamic information.

7. The system of claim 1 wherein generating the virtual mobility test includes providing auditory or haptic feedback to the user when a feedback condition occurs, wherein the feedback condition includes a collision between a user and an obstacle in the virtual mobility test, a user leaves a designated path or course associated with the virtual mobility test, or a predetermined amount of progress has not occurred in a predetermined amount of time.

8. The system of claim 1 generating the virtual mobility test includes capturing the data from the body movement detection sensors and using the data to output a video of the user’s progress through the virtual mobility test.

9. The system of claim 1 wherein the objects in the virtual mobility test may include a tile, an obstacle, a box obstacle, a step-over obstacle, a hanging or swinging obstacle, a floating obstacle, a starting line, a finish line, a finish flag, a guide arrow, or a button obstacle.

10. A method, the method comprising: configuring a virtual mobility test for testing visual function of a user; generating the virtual mobility test; and analyzing performance of the user during the virtual mobility test for determining the visual function of the user based on user interaction with objects in the virtual mobility test using data obtained from body movement detection sensors.

11. The method of claim 10 wherein configuring the virtual mobility test includes configuring the virtual mobility test based on the user or a vision condition; or wherein configuring the virtual mobility test includes configuring the virtual mobility test to test a right eye, a left eye, or both eyes.

12. The method of claim 10 wherein analyzing the performance of the user includes evaluating the effectiveness of a gene therapy or related treatment for a vision condition of the user, measuring one or more symptoms of a vision condition, or diagnosing the user with a vision condition.

13. The method of claim 10 wherein configuring the virtual mobility test includes configuring luminance, shadow, color, contrast, gradients of contrast or color on the surface of the objects, reflectance or color of borders of the objects, or a lighting condition associated with one or more of the objects in the virtual mobility test based on configuration information.

14. The method of claim 10 wherein configuring the virtual mobility test includes: configuring the height of one or more of the objects in the virtual mobility test and/or configuring the size of one or more of the objects in the virtual mobility test based on configuration information.

15. The method of claim 14 wherein the configuration information includes the height or other attributes of the user, condition-based information; user-inputted information, operator-inputted information, or dynamic information.

16. The method of claim 10 wherein generating the virtual mobility test includes providing auditory or haptic feedback to the user when a feedback condition occurs, wherein the feedback condition includes a collision between the user and one or more of the objects in the virtual mobility test, the user leaves a designated path or course associated with the virtual mobility test, or a predetermined amount of progress has not occurred in a predetermined amount of time.

17. The method of claim 10 generating the virtual mobility test includes capturing the data from body movement detection sensors and using the data to output a video of the user’s progress through the virtual mobility test.

18. The method of claim 10 wherein the objects in the virtual mobility test may include a tile, an obstacle, a box obstacle, a step-over obstacle, a hanging or swinging obstacle, a floating obstacle, a starting line, a finish line, a finish flag, a guide arrow, or a button obstacle.

19. A non-transitory computer readable medium having stored thereon executable instructions that when executed by at least one processor of a computer cause the computer to perform steps comprising: configuring a virtual mobility test for testing visual function of a user; generating the virtual mobility test; and analyzing performance of the user during the virtual mobility test for determining the visual function of the user based on user interaction with objects in the virtual mobility test using data obtained from body movement detection sensors.

20. The non-transitory computer readable medium of claim 19 wherein configuring the virtual mobility test includes configuring the virtual mobility test based on the user or a vision condition; or wherein configuring the virtual the virtual mobility test includes configuring the virtual mobility test to test a right eye, a left eye, or both eyes.
